(19) United States
(12) Patent Application Publication
(10) Pub. No.: US 2021/0156866 A1
(43) Pub. Date: May 27, 2021

(54) DETECTION OF IMMUNE CHECKPOINT MOLECULES BY DEGLYCOSYLATION

(71) Applicant: Board of Regents, The University of Texas System, Austin, TX (US)

(72) Inventors: Heng-Huan LEE, Houston, TX (US); Ying-Nai WANG, Houston, TX (US); Mien-Chie HUNG, Houston, TX (US)

(73) Assignee: Board of Regents, The University of Texas System, Austin, TX (US)

(21) Appl. No.: 16/972,259

(22) PCT Filed: Jun. 7, 2019
(86) PCT No.: PCT/US19/36073

§ 371 Date: Dec. 4, 2020

Related U.S. Application Data
(60) Provisional application No. 62/681,929, filed on Jun. 7, 2018.

(51) Int. Cl. G01N 33/574; C12N 9/80; A61K 45/06; C07K 16/28; G01N 33/563

(52) U.S. Cl. CPC G01N 33/57492; C12N 9/80; A61K 45/06; G01N 2800/52; C07K 16/2827; G01N 33/563; C12Y 305/01052; C07K 16/2818

(57) ABSTRACT

Provided herein are methods of detecting glycosylated proteins, such as immune checkpoint proteins. Further provided are methods of treating cancer, such as by administering immune checkpoint inhibitors.

This application claims the benefit of U.S. Provisional Patent Application No. 62/681,929 filed Jun. 7, 2018, the entirety of which is incorporated herein by reference.

### BACKGROUND

### 1. Field

The present invention relates generally to the field of molecular biology. More particularly, it concerns the detection of glycosylated proteins, such as immune checkpoint molecules.

### 2. Description of Related Art

The Food and Drug Administration (FDA) approval of immune checkpoint inhibitors has dramatically changed treatment paradigms for patients with advanced-stage or metastatic non-small cell lung cancer (NSCLC). Despite significant improvements in survival, the majority of NSCLC patients fail to respond to checkpoint inhibitors, notably antibodies targeting programmed death-1 (PD-1) and programmed death ligand-1 (PD-L1), and uncertainties remain regarding how best to use these therapies in clinical practice. Given the risk of immune-related and other adverse effects associated with treatment, there is a need to identify proteins to predict which patients will and will not benefit (Matthew et al., 2017).

Checkpoint inhibitors block inhibitory T cell signaling, thereby leading to an endogenous antitumor immune response. PD-1 is a transmembrane immunoregulatory molecule responsible for the negative regulation of T cell activation and peripheral tolerance. It is expressed on T cells, B cells, and natural killer (NK) cells and binds to its ligands PD-L1 and PD-L2. Expression of PD-L1 rarely occurs on normal tissues but is prevalent on antigen presenting cells (APCs) and tumor cells in numerous solid malignancies including NSCLC. There is constitutive expression of PD-L1 on tumor cells, which occurs during oncogenic processes in a state of chronic antigen presentation, and inducible expression of PD-L1 at the tumor site in the presence of pro-inflammatory cytokines such as interferon-gamma.

Over the past several years, studies have demonstrated improved outcomes with checkpoint inhibitors compared to conventional chemotherapy in advanced NSCLC. The expression of PD-L1 on tumor cell membranes via immunohistochemistry (IHC) has been most widely studied for use with anti-PD-1/PD-L1 therapy in this setting. While its role as a companion or complementary diagnostic assay in the refractory setting has been studied extensively, an even more important role for PD-L1 has emerged for selecting patients for upfront treatment. With numerous trials focusing on first-line immunotherapy, patient selection is more important than ever. However, there are limitations to using PD-L1 as a predictive biomarker in NSCLC due to a lack of accurate diagnostic assays that can measure PD-L1 expression.

Immunohistochemical (IHC) staining of membrane proteins including immune checkpoint molecules is a primary method to provide critical information for clinical diagnosis and therapeutic response. Accurate IHC staining of target proteins is critical for precision medicine and immunotherapy. Accumulating evidence shows that immunotherapy through immune checkpoint blockade such as anti-PD-L1 therapy significantly improves patient survival rate and clinical outcome among different cancer types. However, there is no direct biomarker available for anti-PD-L1 therapy since PD-L1 IHC staining readout is not associated with patient response after anti-PD-L1 therapy, where it remains unclear if this discrepancy comes from certain unknown biological mechanism or technical issue for detection (Garon et al. 2015; Ma et al., 2016). Thus, there is an unmet need for improved detection methods for measuring the expression level of PD-L1 to better predict clinical response to immune checkpoint inhibitors.

### SUMMARY

In a first embodiment, the present disclosure provides an in vitro method for detecting the level of an immune checkpoint protein comprising obtaining a fixed sample; deglycosylating proteins in said sample; contacting said sample with an anti-immune checkpoint protein antibody; and measuring the binding of the antibody to said immune checkpoint protein, thereby detecting the level of said immune checkpoint protein. In particular aspects, the subject is human.

In some aspects, the fixed sample is a formalin fixed sample or paraformaldehyde fixed sample. In certain aspects, the formalin fixed sample is further defined as a formalin-fixed paraffin-embedded (FFPE) sample. In some aspects, the fixed sample is isolated from saliva, blood, urine, normal tissue, or tumor tissue. In specific aspects, the fixed sample does not comprise live cells.

In certain aspects, the immune checkpoint protein is PD-L1, PD-L2, TIM-3, B7-H3, B7-H4, VISTA, CD40, PD-1, CTLA-4, or OX-40L. In some aspects, the anti-immune checkpoint protein antibody is an anti-PD-L1 antibody.

In some aspects, deglycosylating comprises contacting the fixed sample with a deglycosylation enzyme. In certain aspects, the deglycosylation enzyme removes N-linked glycosylation. In particular aspects, the deglycosylation enzyme is peptide-N-glycosidase (PNGase F). In some aspects, the sample is incubated with PNGase F for 12-16 hours, such as 12, 13, 14, 15, or 16 hours. In particular aspects, the sample is incubated with PNGase F at a concentration of 1-10%, such as 1%, 2%, 3%, 4%, 5%, 6%, 7%, 8%, 9%, or 10%.

In certain aspects, measuring comprises performing immunoblotting, immunohistochemistry, ELISA, or immunofluorescence. In particular aspects, measuring comprises performing immunohistochemistry.

In additional aspects, the method further comprises administering an anti-cancer therapy to a subject identified to have an increased level of the immune checkpoint protein as compared to a control sample. In some aspects, the subject has been previously determined to have no or low expression of the immune checkpoint protein using a non-deglycosylated sample. In certain aspects, the anti-cancer therapy is chemotherapy, radiotherapy, gene therapy, surgery, hormonal therapy, anti-angiogenic therapy or immunotherapy. In some aspects, the anti-cancer therapy is immunotherapy. In particular aspects, the immunotherapy is an immune checkpoint inhibitor. In some aspects, the immune checkpoint inhibitor is a PD-L inhibitor. In some aspects, the PD-L1 inhibitor is atezolizumab, avelumab, durvalumab, BMS-936559, or CK-301. In certain aspects, the immune checkpoint inhibitor is a PD-1 inhibitor. In some aspects, the PD-1 inhibitor is pembrolizumab or nivolumab. In some aspects, the immune checkpoint inhibitor is a CTLA-4 inhibitor. In specific aspects, the CTLA-4 inhibitor is ipilimumab or tremelimumab.

In some aspects, the immune checkpoint inhibitor is administered intravenously, intraperitoneally, intratracheally, intratumorally, intramuscularly, endoscopically, intralesionally, percutaneously, subcutaneously, regionally, or by direct injection or perfusion. In particular aspects, the immune checkpoint inhibitor is administered intravenously.

In some aspects, the subject has an immune checkpoint-associated disease, such as cancer, diabetes, neurodegenerative disease, or rheumatic disease. In particular aspects, the cancer is breast cancer, lung cancer, colon cancer, prostate cancer, pancreatic cancer, melanoma, hepatocellular carcinoma, renal cell carcinoma, urothelial cell carcinoma, or Hodgkin's lymphoma.

A further embodiment provides a pharmaceutical composition comprising an immune checkpoint inhibitor for use in a subject determined to have an increased level of said immune checkpoint protein according to the method of the embodiments and aspects thereof. In some aspects, the immune checkpoint protein is PD-L1, PD-L2, TIM-3, B7-H3, B7-H4, VISTA, CD40, PD-1, CTLA-4, or OX-40L. In certain aspects, the immune checkpoint protein is PD-L1. In some aspects, the immune checkpoint inhibitor is PD-L1 inhibitor. In specific aspects, the PD-L1 inhibitor is atezolizumab, avelumab, durvalumab, BMS-936559, or CK-301. In some aspects, the immune checkpoint inhibitor is a PD-1 inhibitor. In certain aspects, the PD-1 inhibitor is pembrolizumab or nivolumab. In some aspects, the immune checkpoint inhibitor is a CTLA-4 inhibitor. In certain aspects, the CTLA-4 inhibitor is ipilimumab or tremelimumab.

Another embodiment provides a composition comprising an effective amount of an immune checkpoint inhibitor for the treatment of cancer in a subject, wherein the subject has been determined to have an increased level of said immune checkpoint protein according to the methods of the present embodiments and aspects thereof. In some aspects, the immune checkpoint protein is PD-L1, PD-L2, TIM-3, B7-H3, B7-H4, VISTA, CD40, PD-1, CTLA-4, or OX-40L. In certain aspects, the immune checkpoint protein is PD-L1. In some aspects, the immune checkpoint inhibitor is PD-L1 inhibitor. In specific aspects, the PD-L1 inhibitor is atezolizumab, avelumab, durvalumab, BMS-936559, or CK-301. In some aspects, the immune checkpoint inhibitor is a PD-1 inhibitor. In certain aspects, the PD-1 inhibitor is pembrolizumab or nivolumab. In some aspects, the immune checkpoint inhibitor is a CTLA-4 inhibitor. In certain aspects, the CTLA-4 inhibitor is ipilimumab or tremelimumab.

A further embodiment provides a method of predicting response to an immune checkpoint inhibitor in a subject having cancer comprising measuring the level of the immune checkpoint protein according to the method of the present embodiments and aspects thereof in a sample obtained from said subject, wherein if the sample has increased expression of the immune checkpoint protein, then the patient is predicted to have a favorable response to the immune checkpoint inhibitor.

In some aspects, the immune checkpoint protein is PD-L1, PD-L2, TIM-3, B7-H3, B7-H4, VISTA, CD40, PD-1, CTLA-4, or OX-40L. In certain aspects, the immune checkpoint protein is PD-L. In some aspects, the immune checkpoint inhibitor is a PD-L1 inhibitor. In some aspects, the PD-L1 inhibitor is atezolizumab, avelumab, durvalumab, BMS-936559, or CK-301. In some aspects, the immune checkpoint inhibitor is a PD-1 inhibitor. In certain aspects, the PD-1 inhibitor is pembrolizumab or nivolumab. In some aspects, the immune checkpoint inhibitor is a CTLA-4 inhibitor. In certain aspects, the CTLA-4 inhibitor is ipilimumab or tremelimumab.

In certain aspects, measuring comprises performing immunoblotting, immunohistochemistry, ELISA, or immunofluorescence. In some aspects, further comprising administering the immune checkpoint inhibitor to the subject predicted to have a favorable response. In certain aspects, the immune checkpoint inhibitor is administered intravenously, intraperitoneally, intratracheally, intratumorally, intramuscularly, endoscopically, intralesionally, percutaneously, subcutaneously, regionally, or by direct injection or perfusion.

In yet another embodiment, there is provided a method of treating an immune checkpoint-associated disease, such as cancer, diabetes, neurodegenerative disease, or rheumatic disease, in a subject comprising administering an effective amount of an immune checkpoint inhibitor to the subject, wherein the subject has been determined to have an increased level of an immune checkpoint protein according to the method of the present embodiments and aspects thereof. In some aspects, the subject is human.

In some aspects, the immune checkpoint protein is PD-L1, PD-L2, TIM-3, B7-H3, B7-H4, VISTA, CD40, PD-1, CTLA-4, or OX-40L. In certain aspects, the immune checkpoint protein is PD-L1. In some aspects, the immune checkpoint inhibitor is PD-L1 inhibitor. In specific aspects, the PD-L1 inhibitor is atezolizumab, avelumab, durvalumab, BMS-936559, or CK-301. In some aspects, the immune checkpoint inhibitor is a PD-1 inhibitor. In particular aspects, the PD-1 inhibitor is pembrolizumab or nivolumab. In some aspects, the immune checkpoint inhibitor is a CTLA-4 inhibitor. In particular aspects, the CTLA-4 inhibitor is ipilimumab or tremelimumab.

In some aspects, the immune checkpoint inhibitor is administered intravenously, intraperitoneally, intratracheally, intratumorally, intramuscularly, endoscopically, intralesionally, percutaneously, subcutaneously, regionally, or by direct injection or perfusion.

In certain aspects, the cancer is breast cancer, lung cancer, colon cancer, prostate cancer, pancreatic cancer, melanoma, hepatocellular carcinoma, renal cell carcinoma, urothelial cell carcinoma, or Hodgkin's lymphoma.

In additional aspects, the method further comprises administering an additional anti-cancer therapy. In some aspects, the additional anti-cancer therapy is chemotherapy, radiotherapy, gene therapy, surgery, hormonal therapy, anti-angiogenic therapy or immunotherapy. In certain aspects, the additional anti-cancer therapy is administered concurrently with the immune checkpoint inhibitor.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1H Removal of N-linked glycosylation enhances anti-PD-L1 signal in human cancer cells in a variety of bioassays. (A and B) Immunofluorescence confocal microscopy of BT-549 (A) and A549 (B), treated with (+) or without (−) PNGase F (5%) overnight at 37° C. stained with DAPI and an anti-PD-L1 antibody (Abeam, ab58810). Bar, 10 μm. Quantification is shown right. Data are representative of 3 independent experiments, randomly chosen in 3 different fields. (C and D) ELISA of Con A (C) and PD-L1 (Abcam, ab205921, clone 28-8 mAb) (D) levels in BT-549 cells treated with increasing concentrations of PNGase F (1, 2, 5%) overnight at 37° C. for comparison with cells without treatment (0%). (E) ELISA of PD-L1 level (Abcam, ab205921, clone 28-8 mAb) in lung cancer cells treated with (+) PNGase F (1%) overnight at 37° C. for comparison with cells without (−) treatment. Negative control, secondary Ab only control. (F) Left: saturation binding assay of A549 cell lysates binding to anti-PD-L1 clone 28-8 mAb. Right: scatchard plot of cell number binding to anti-PD-L1 antibody transformed from Kd values determined by saturation binding assay. (G) Representative images (left) and quantification (right) of H-score of IHC staining for BLBC (BT-549, BT-20, and MDA-MB-231) and non-BLBC (MCF-7) cancer cell blocks treated with or without PNGase F (5%) overnight at 37° C. Bar, 50 sm. (H) Representative images (top) and quantification (bottom) of H-score of IHC staining for a panel of lung cancer cell blocks treated with or without PNGase F (5%) overnight at 37° C. Bar, 50 sm. All error bars represent mean±SD. *p<0.05, **p<0.01, ***p<0.001, Student's t test.

FIGS. 2A-2G: Deglycosylation significantly enhances anti-PD-L1 signal in a major population of patient samples in human tumor tissue microarray. (A) H-score values representing PD-L1 protein expression treated with or without PNGase F (5%) overnight at 37° C. from IHC staining of a human multi-organ carcinoma tissue microarray (TMA) (n=200). Wilcoxon signed-rank test. (B) A pie chart highlighting the fold change of H-score after N-linked glycosylation removal through PNGase F treatment from (A). (C) Two representative cases of IHC staining from (A). Bar, 50 μm. (D) H-score values representing PD-L1 protein expression treated with or without PNGase F (5%) overnight at 37° C. from IHC staining of a human lung cancer TMA (n=149). Wilcoxon signed-rank test. (E) A pie chart highlighting the fold change of H-score after N-linked glycosylation removal through PNGase F treatment from (D). (F) Two representative cases of IHC staining from (D). Bar, 50 μm. (G) The average population of three individual cohorts of lung cancer patients (total n=233) expressing PD-L1 positive cells (PD-L1 TPS; %) from the indicated cutoffs without and with deglycosylation (abbreviated to deglyco). Error bars represent mean±SD.

FIGS. 3A-3G: Improved PD-L1 detection after deglycosylation is associated with response to anti-PD-1/PD-L1 therapy. (A) H-score representing PD-L1 protein expression treated with or without PNGase F (5%) overnight at 37° C. from IHC staining of the archived FFPE tumor tissue blocks before treatments from multiple types of cancer patients who received or are undergoing anti-PD-1/PD-L1 immunotherapy (n=95). Wilcoxon signed-rank test. (B) A pie chart highlighting the fold change in H-score after N-linked glycosylation removal through PNGase F treatment from (A). (C) Representative cases of IHC staining from (A). Bar, 50 μm. (D) Pearson correlation test between H-score representing PD-L1 protein expression and the corresponding PFS from anti-PD-1/PD-L1 therapy in patient tissue slides treated with or without PNGase F from (A). (E) Pearson correlation test between the percentage of PD-L1 positive cells (TPS) and the corresponding PFS from anti-PD-1/PD-L1 therapy in patient tissue slides treated with or without PNGase F from (A). (F and G) Pearson correlation test between PD-L1 H-score (F) or PD-L1 TPS (G) and the corresponding OS from anti-PD-1/PD-L1 therapy in patient tissue slides treated with or without PNGase F from (A) (n=49 with the OS available).

FIGS. 4A-4D: Increased PD-L1 signal after deglycosylation is beneficial to therapeutic selection. (A and B) Pearson correlation test between PD-L1 H-score (A) or PD-L1 TPS (B) and the corresponding PFS from anti-PD-1/PD-L1 therapy in lung cancer patient tissue slides (n=44) treated with or without PNGase F from FIG. 3A. (C) The PFS of lung cancer patients expressing PD-L1 TPS in the indicated cutoffs without or with deglycosylation (abbreviated to deglyco). n=12 for group (1). n=7 for group (2). n=25 for group (3). (D) The PFS of lung cancer patients expressing PD-L1 TPS from <1% in the indicated cutoffs without or with deglycosylation (abbreviated to deglyco). n=10 for group (4). n=5 for group (5). n=3 for group (6). Error bars (lines) represent mean±SD. *p<0.05, **p<0.01, ***p<0.001, NS, not significant, Student's t test.

FIGS. 5A-5D: Antigen retrieval by protein deglycosylation improves predictive ability of PD-L1 as a biomarker for immunotherapy. (A and B) The PFS of cancer patient samples processed without (A) or with (B) deglycosylation by PNGase F treatment. Cases with H-score equal to or higher than the median value of total 95 cases (H-score=57.5) were considered as high expression and those with H-score less than the median value as low expression. (C and D) The PFS of cancer patient samples processed without (C) or with (D) deglycosylation by PNGase F treatment. Cases with H-score equal to or higher than the median value of individual group [H-score=40 in the group of without glycosylation (C) and H-score=90 in the group of with glycosylation (D), respectively] were considered as high expression and those with H-score less than the respective median value as low expression. Cohort size for each group is indicated. p values were determined by Log-rank (Mantel-Cox) test. Hazard Ratio (HR) and 95% confidence interval (CI) were determined by Mantel-Haenszel method. (E and F) Illustration of computed tomography (CT) scan and chest X-ray of case 6 (E) and case 11 (F) from FIG. 3A, pre- and post-anti-PD-1 (nivolumab) immunotherapy. (G) A proposed model of PD-L1 antigen retrieval through sample deglycosylation. In brief, the glycan structure of PD-L1 hinders antibody-based detection targeting the PD-L1 antigen. Sample deglycosylation increases homogeneity of PD-L1 and more accurately assesses PD-L1 expression to allow better estimation of PD-L1 levels to prevent false-negative readouts in clinical settings.

FIGS. 6A-6F: Anti-PD-L1 signal is enhanced after deglycosylation in human cancer cells in immunofluorescence and ELISA-based assays. Related to FIG. 1. (A) Cell lysates of lung cancer cells treated with (+) or without (−) PNGase F (5%) overnight at 37° C., and immunoblotting (IB) with the indicated antibodies. Anti-PD-L1 antibody for IB, Cell Signaling (13684). Asterisk indicates non-glycosylated PD-L1. (B) IB of non-BLBC and BLBC cells with the indicated antibodies. (C) Cell lysates of BLBC cells treated with (+) or without (−) PNGase F (5%) overnight at 37° C., and IB with the indicated antibodies. (D) Immunofluorescence confocal microscopy of H1299, treated with (+) or without (−) PNGase F (5%) overnight at 37° C. stained with DAPI and an anti-PD-L1 antibody (Abcam, ab58810). Bar, 10 sm. Quantification is shown right. Data are representative of 3 independent experiments, randomly chosen in 3 different fields. (E) Left: saturation binding assay of H1299 cell lysates binding to anti-PD-L1 antibody (clone 28-8). Right: scatchard plot of cell number binding to anti-PD-L1 antibody transformed from Kd values determined by saturation binding assay. (F) ELISA of PD-L1 level by an anti-PD-L1 antibody atezolizmab (MDACC) in A549 and H1299 cells treated with (+) PNGase F (1%) overnight at 37° C. for comparison with cells without (−) treatment. Negative control, secondary Ab only control. All error bars represent mean SD. *p<0.05, **p<0.01, Student's t test.

FIGS. 7A-7E: Anti-PD-L1 signal is enhanced after removal of N-linked glycosylation in a major population of patient samples in different cancer types. Related to FIG. 2. (A) Individual analysis of five cohorts in multi-organ carcinoma TMA from FIG. 2A, containing 40 cases each of breast invasive ductal carcinoma, lung squamous cell carcinoma, colon adenocarcinoma, prostate adenocarcinoma, and pancreas adenocarcinoma. (B) H-score values representing PD-L1 protein expression from IHC staining of a human rectal cancer TMA (n=92) processed with or without deglycosylation by PNGase F (5%) pretreatment. Results were analyzed by the Wilcoxon signed-rank test. (C) A pie chart highlighting the fold change of H-score after N-linked glycosylation removal through PNGase F treatment from (B). (D) Two representative cases of IHC staining from (B). Bar, 50 μm. (E) Representative images of PD-L1 IHC staining in the lung cancer tumor microarray (Biomax, #NSC151). Samples displayed varying percentages of the stained cells spanning a wide range from negative (0%) to strongly positive (100%) staining of the tumor cells. Bar, 50 μm. Inset: PD-L1 membrane staining (arrows); bar, 20 μm.

FIGS. 8A-8H: Removal of N-linked glycosylation improves PD-L1 detection in clinical samples and correlation with patient responses to anti-PD-1/PL1 therapy. Related to FIG. 3. (A) The percentage of PD-L1 positive signals in tumor cells (TPS; tumor proportion score; % positive cells) treated with or without PNGase F (5%) overnight at 37° C. from IHC staining of patient tissue slides shown in FIG. 3A (n=95). Wilcoxon signed-rank test. (B) A pie chart highlighting the fold change in PD-L1 TPS after N-linked glycosylation removal by PNGase F treatment from (A). (C and D) Correlation between PD-L1 H-score (C) or PD-L1 TPS (D) and the corresponding progression-free survival (PFS) from nivolumab therapy (n=39) in patient tissue slides treated with or without PNGase F from FIG. 3A. (E and F) Correlation between PD-L1 H-score (E) or PD-L1 TPS (F) and the corresponding PFS from anti-PD-1 therapy (nivolumab, pembrolizumab, and camrelizumab; n=75) in patient tissue slides treated with or without PNGase F from FIG. 3A. (G and H) Correlation between PD-L1 H-score (G) or PD-L1 TPS (H) and the corresponding PFS from anti-PD-L1 therapy (atezolizumab and durvalumab; n=12) in patient tissue slides treated with or without PNGase F from FIG. 3A. One-tailed p values are shown, Pearson correction test.

FIGS. 9A-9K: Sample deglycosylation enhances PD-L1 detection in a small fraction of tumor associated lymphocytes. Related to FIG. 3. (A) Cell lysates of human immune cells, including Jurkat (T lymphocytes) and THP1 (monocytes), were treated with (+) or without (−) PNGase F (5%) overnight at 37° C., and subjected to IB with PD-L1 antibody (Cell Signaling, 13684). *, non-glycosylated PD-L. (B) Jurkat and THP1 cells were treated with or without PNGase F (%) overnight at 37° C., and subjected to ELISA to determine the PD-L1 levels for comparison. Error bars represent mean±SD. *p<0.05, Student's t test. (C) The percentage of PD-L1 positive signals in immune cells (% PD-L1+immune cells) treated with or without PNGase F (5%) overnight at 37° C. from IHC staining of patient tissue slides shown in FIG. 3A (n=46 containing tumor-associated immune cells). Wilcoxon signed-rank test. (D) A pie chart highlighting the fold change in the percentage of PD-L1-immune cells after N-linked glycosylation removal by PNGase F treatment from (C). (E) Two representative cases of IHC staining from (C). PD-L1+tumor cells (TPS; tumor proportion score), white arrows; PD-L1+immune cells, red arrows. Bar, 50 μm. (F) The percentage of PD-L1 positive signals in tumor cells (TPS; % positive cells) treated with or without PNGase F (5%) overnight at 37° C. from IHC staining from (C). Wilcoxon signed-rank test. (G) A pie chart highlighting the fold change in PD-L1 TPS after N-linked glycosylation removal by PNGase F treatment from (F). (H) Correlation between PD-L1 TPS and the corresponding PFS from anti-PD-1/PD-L1 therapy in patient tissue slides treated with or without PNGase F from (F). Pearson correction test; one-tailed. (I) PD-L1 positive signals in both tumor and immune cells (CPS; combined positive score) treated with or without PNGase F (5%) overnight at 37° C. from IHC staining from (C). Wilcoxon signed-rank test. (J) A pie chart highlighting the fold change in PD-L1 CPS after N-linked glycosylation removal by PNGase F treatment from (I). (K) Correlation between PD-L1 CPS and the corresponding PFS from anti-PD-1/PD-L1 therapy in patient tissue slides treated with or without PNGase F from (I). Pearson correction test; one-tailed.

FIGS. 10A-10H: Antigen retrieval by protein deglycosylation improves the utility of PD-L1 as a predictive biomarker for immunotherapy. Related to FIG. 5. (A and B) The overall survival (OS) of cancer patient samples processed without (A) or with (B) deglycosylation by PNGase F treatment. Cases with H-score equal to or higher than the median value of total 49 cases (H-score=15.0) were considered as high expression and those with H-score less than the median value as low expression. (C and D) The OS of cancer patient samples processed without (C) or with (D) deglycosylation by PNGase F treatment. Cases with H-score equal to or higher than the median value of individual group [H-score=8.0 in the group of without glycosylation (C) and H-score=30.0 in the group of with glycosylation (D), respectively] were considered as high expression and those with H-score less than the respective median value as low expression. (E and F) The OS of cancer patient samples processed without (E) or with (F) deglycosylation by PNGase F treatment. Cases with PD-L1 TPS equal to or higher than the median value of total 49 cases (PD-L1 TPS=30%) were considered as high expression and those with PD-L1 TPS less than the median value as low expression. (G and H) The OS of cancer patient samples processed without (G) or with (H) deglycosylation by PNGase F treatment. Cases with PD-L1 TPS equal to or higher than the median value of individual group [PD-L1 TPS=15% in the group of without glycosylation (G) and PD-L1 TPS=40% in the group of with glycosylation (H), respectively] were considered as high expression and those with PD-L1 TPS less than the respective median value as low expression. Cohort size for each group is indicated. p values were determined by Log-rank (Mantel-Cox) test. Hazard Ratio (HR) and 95% confidence interval (CI) were determined by Mantel-Haenszel method.

### DESCRIPTION OF ILLUSTRATIVE EMBODIMENTS

Glycosylation is well-known to be important for membrane protein biogenesis and function. As surface antigens, immune checkpoint proteins like PD-L1 are known to be glycosylated and the glycan moiety is important for its biological function. However, there are no studies showing that glycosylation can interrupt anti-PD-L1 antibody binding to PD-L1 to cause inconsistent detection. Hence, there is no method reported to use protein deglycosylation to improve anti-PD-L1 antibody binding capability. There is currently no method for protein deglycosylation developed for a general application in antibody-based detection of surface antigens, such as immune checkpoint molecules.

In the present studies, it was hypothesized that glycans may play a negative role for interaction between the anti-PD-L1 antibody and the PD-L1 antigen. The present studies found that N-linked glycosylation removal can improve anti-PD-L1 detection and pathological correlation with therapeutic outcome. The deglycosylation method significantly enhanced PD-L1 signal compared with the conventional method, in three different tissue microarrays (TMAs).

Accordingly, in certain embodiments, the present disclosure provides methods of detecting proteins, such as immune checkpoint proteins, by removing glycosylation to facilitate antibody recognition during evaluation of the antigen amount for both fundamental and clinical applications. The deglycosylated protein may then be detect by antibody-based detection methods, such as immunoblotting, quantitative ELISA, immunofluorescence (IP) imaging and IHC staining. The method may be applied for research, pharmaceutical, and clinical (e.g., diagnostic) purposes. Specifically, the present methods provide improved prediction of patient response for precision medicine in immune oncology.

Heavy glycosylation of PD-L1 hinders its detection by the PD-L1 antibodies and could lead to inaccurate readout from a variety of bioassays. In certain aspects, the removal of PD-L1 N-linked glycosylation by enzymatic digestion of tissue samples in the present methods can be used to increase homogeneity of target proteins and quantitatively facilitate antibody-based detection for a more precise estimation of PD-L1 levels to prevent false-negative readouts in clinical settings. Since cell surface proteins are frequently N-link glycosylated at different levels, this deglycosylation method can be used as a general approach to decrease antigen heterogeneity and eliminate structural hindrance prior to antibody detection with great potential to improve biomedical research and personalized medicine.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

As used herein, the term “subject” refers to a human or non-human mammal or animal. Non-human mammals include livestock animals, companion animals, laboratory animals, and non-human primates. Non-human subjects also specifically include, without limitation, chickens, horses, cows, pigs, goats, dogs, cats, guinea pigs, hamsters, mink, and rabbits. In some embodiments of the invention, a subject is a patient. As used herein, a “patient” refers to a subject who is under the care of a physician or other health care worker, including someone who has consulted with, received advice from or received a prescription or other recommendation from a physician or other health care worker.

The term “effective,” as that term is used in the specification and/or claims, means adequate to accomplish a desired, expected, or intended result. “Effective amount,” “therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

The term “antibody” herein is used in the broadest sense and specifically covers monoclonal antibodies (including full length monoclonal antibodies), polyclonal antibodies, multi-specific antibodies (e.g., bispecific antibodies), and antibody fragments so long as they exhibit the desired biological activity.

The term “determining an expression level” as used herein means the application of a gene specific reagent such as a probe, primer or antibody and/or a method to a sample, for example a sample of the subject and/or a control sample, for ascertaining or measuring quantitatively, semi-quantitatively or qualitatively the amount of a gene or genes, for example the amount of mRNA. For example, a level of a gene can be determined by a number of methods including for example immunoassays including for example immunohistochemistry, ELSA, Western blot, immunoprecipitation and the like, where a biomarker detection agent such as an antibody for example, a labeled antibody, specifically binds the biomarker and permits for example relative or absolute ascertaining of the amount of polypeptide biomarker, hybridization and PCR protocols where a probe or primer or primer set are used to ascertain the amount of nucleic acid biomarker, including for example probe based and amplification based methods including for example microarray analysis, RT-PCR such as quantitative RT-PCR, serial analysis of gene expression (SAGE). Northern Blot, digital molecular barcoding technology, for example Nanostring:nCounter™ Analysis, and TaqMan quantitative PCR assays. Other methods of mRNA detection and quantification can be applied, such as mRNA in situ hybridization in formalin-fixed, paraffin-embedded (FFPE) tissue samples or cells. This technology is currently offered by the QuantiGene® ViewRNA (Affymetrix), which uses probe sets for each mRNA that bind specifically to an amplification system to amplify the hybridization signals; these amplified signals can be visualized using a standard fluorescence microscope or imaging system. This system for example can detect and measure transcript levels in heterogeneous samples; for example, if a sample has normal and tumor cells present in the same tissue section. As mentioned, TaqMan probe-based gene expression analysis (PCR-based) can also be used for measuring gene expression levels in tissue samples, and for example for measuring mRNA levels in FFPE samples. In brief, TaqMan probe-based assays utilize a probe that hybridizes specifically to the mRNA target. This probe contains a quencher dye and a reporter dye (fluorescent molecule) attached to each end, and fluorescence is emitted only when specific hybridization to the mRNA target occurs. During the amplification step, the exonuclease activity of the polymerase enzyme causes the quencher and the reporter dyes to be detached from the probe, and fluorescence emission can occur. This fluorescence emission is recorded and signals are measured by a detection system; these signal intensities are used to calculate the abundance of a given transcript (gene expression) in a sample.

The term “sample” as used herein includes any biological specimen obtained from a patient. Samples include, without limitation, whole blood, plasma, serum, red blood cells, white blood cells (e.g., peripheral blood mononuclear cells), ductal lavage fluid, nipple aspirate, lymph (e.g., disseminated tumor cells of the lymph node), bone marrow aspirate, saliva, urine, stool (i.e., feces), sputum, bronchial lavage fluid, tears, fine needle aspirate (e.g., harvested by fine needle aspiration that is directed to a target, such as a tumor, or is random sampling of normal cells, such as periareolar), any other bodily fluid, a tissue (e.g., tumor tissue) such as a biopsy of a tumor (e.g., needle biopsy) or a lymph node (e.g., sentinel lymph node biopsy), and cellular extracts thereof. In some embodiments, the sample is whole blood or a fractional component thereof such as plasma, serum, or a cell pellet.

As used herein, a “fixed” sample refers to a sample which has undergone preservation. The fixation can terminate any biochemical reactions and increase the tissue's stability. Chemical fixation methods can include subjecting the sample to aldehydes, such as formaldehyde or glutaraldehyde, or alcohols, such as methanol or ethanol.

The terms “increased”, “elevated”, “overexpress”, “overexpression”, “overexpressed”, “up-regulate”, or “up-regulated” interchangeably refer to a biomarker that is present at a detectably greater level in a biological sample, e.g. plasma, from a patient with cancer, in comparison to a biological sample from a patient without cancer. The term includes overexpression in a sample from a patient with cancer due to transcription, post-transcriptional processing, translation, post-translational processing, cellular localization (e.g, organelle, cytoplasm, nucleus, cell surface), and RNA and protein stability, as compared to a sample from a patient without cancer. Overexpression can be detected using conventional techniques for detecting mRNA (i.e., RT-PCR, PCR, hybridization) or proteins (i.e., ELISA, immunohistochemical techniques, mass spectroscopy, Luminex® xMAP technology). Overexpression can be 10%, 20%, 30%, 40%, 50%, 60%, 70%, 80%, 90% or more in comparison to a sample from a patient without cancer. In certain instances, overexpression is 1-fold, 2-fold, 3-fold, 4-fold 5, 6, 7, 8, 9, 10, or 15-fold or more higher levels of transcription or translation in comparison to a sample from a patient without cancer.

As used herein, the term “detecting” refers to observing a signal from a label moiety to indicate the presence of a biomarker in the sample. Any method known in the art for detecting a particular detectable moiety can be used for detection. Exemplary detection methods include, but are not limited to, spectroscopic, photochemical, biochemical, immunochemical, electrical, optical or chemical methods.

### II. ANTIBODY-BASED DETECTION METHODS

Certain embodiments of the present disclosure concern immunodetection methods for binding, purifying, removing, quantifying or otherwise generally detecting glycosylated proteins, such as proteins with N-linked glycosylation. The molecules may be immune checkpoint molecules, such as PD-L1, or any other protein which is glycosylated, such as membrane proteins.

In some aspects, the present methods comprise deglycosylation of a sample comprising proteins for improved detection of the proteins which are glycosylated. The deglycosylation may be through enzymatic treatment of the sample, such as with the enzyme Peptide-N-Glycosidase (PNGase F; NEB P0704).

Any antibody-based method of detection is contemplated for use with the present methods. For example, the present methods could be used for the detection of immune checkpoint molecules such as by immunoblotting, quantitative ELISA, immunofluorescence (IF) imaging, and IHC staining. The steps of various useful immunodetection methods have been described in the scientific literature, such as, e.g., Nakamura el al. (1987), incorporated herein by reference.

As used herein, sample may refer to a whole organism or a subset of its tissues, cells or component parts. A sample may also refer to a homogenate, lysate, or extract prepared from a whole organism or a subset of its tissues, cells or component parts, or a fraction or portion thereof. Non-limiting examples of samples include urine, blood, cerebrospinal fluid (CSF), pleural fluid, sputum, and peritoneal fluid, bladder washings, secretions, oral washings, tissue samples, touch preps, or fine-needle aspirates. In some embodiments, a sample may be a cell line, cell culture or cell suspension. Preferably, a sample corresponds to the amount and type of expression products present in a parent cell from which the sample was derived. A sample can be from a human or non-human subject. In some embodiments, the sample used for performing antibody-based detection is a formalin fixed paraffin embedded (FFPE) specimen.

The sample may comprise body fluids and tissue samples that include but are not limited to blood, tissue biopsies, spinal fluid, meningeal fluid, urine, alveolar fluid. For those tissue samples in which the cells do not naturally exist in a monolayer, the cells can be dissociated by standard techniques known to those skilled in the art. These techniques include but are not limited to trypsin, collagenase or dispase treatment of the tissue.

Typically, cells are harvested from a sample using standard techniques. For example, cells can be harvested by centrifuging a biological sample such as urine, and resuspending the pelleted cells. Typically, the cells are resuspended in phosphate-buffered saline (PBS). After centrifuging the cell suspension to obtain a cell pellet, the cells can be fixed, for example, in acid alcohol solutions, acid acetone solutions, or aldehydes such as formaldehyde, paraformaldehyde, and glutaraldehyde. For example, a fixative containing methanol and glacial acetic acid in a 3:1 ratio, respectively, can be used as a fixative. A neutral buffered formalin solution also can be used, and includes approximately 10% to 10% of 37-40% formaldehyde in an aqueous solution of sodium phosphate. Slides containing the cells can be prepared by removing a majority of the fixative, leaving the concentrated cells suspended in only a portion of the solution.

The level of expression of the deglycosylated protein may be measured by ELISA, western blotting, mass spectrometry, a capillary immune-detection method, isoelectric focusing, an immune precipitation method or immunohistochemistry. Other methods include of detection include antibody-based optical imaging, ultrasound imaging, MRI imaging. PET imaging, and phototherapy.

In general, the immunobinding methods include obtaining a sample suspected of containing glycosylated protein, polypeptide or peptide, deglycosylating the protein, and contacting the sample with a first antibody under conditions effective to allow the formation of immunocomplexes. The antibody may be linked to a solid support, such as in the form of a column matrix, and the sample suspected of containing the deglycosylated protein may be applied to the immobilized antibody. The unwanted components will be washed from the column, leaving the antigen immunocomplexed to the immobilized antibody, which is then collected by removing the protein from the column.

Contacting the chosen sample with the antibody under conditions effective and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, the deglycosylated antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological or enzymatic tags. U.S. patents concerning the use of such labels include U.S. Pat. Nos. 3,817,837; 3,850,752, 3,939,350; 3,996,345; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

A. Immunohistochemistry (IHC)

The present methods may be used in conjunction with both fresh-frozen and formalin-fixed, paraffin-embedded tissue blocks prepared for study by immunohistochemistry (IHC). The method of preparing tissue blocks from these particulate specimens has been successfully used in previous IHC studies of various prognostic factors, and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and pelleting again by centrifugation; snap-freezing in −70° C. isopentane; cutting the plastic capsule and removing the frozen cylinder of tissue; securing the tissue cylinder on a cryostat microtome chuck; and cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and embedding the block in paraffin; and cutting up to 50 serial permanent sections.

In one exemplary IHC method, the slides may be dried at 40-45° C. in an oven overnight and then incubated at 58-65° C. for 1-3 hours. The slides can then be deparaffinized with xylene and ethanol and hydrated in distilled H₂O. Antigen retrieval can be performed in 10 mM citric acid (pH 6.0) in a microwave for 10 min (2 min 1000 W, 8 min 200 W), cooled down at room temperature for 60 min, and washed with PBS twice. The slides can then be blocked in 3% H₂O₂/methanol for 10 min at room temperature and washed with PBS three times. Normal horse serum or goat serum (10% normal serum in PBS) is applied for 30 min in a humid chamber at room temperature and normal serum is wiped off. The primary antibody in applied in a humid chamber at 4° C. overnight and then washed with PBS three times. The secondary antibody is applied in a humid chamber for 1 hour at room temperature and then washed with PBS three times. Peroxidase conjugated avidin biotin complex is applied in a humid chamber for 1 hour at room temperature and then washed with PBS three times. AEC chromogen substrate is applied for 5-10 min and washed with distilled H₂O three times. The sample is then counterstained with Mayer's hematoxylin for 30 seconds and washed with distilled H₂O three times. Finally, the slides are mounted with aqua-mount (Lerner Laboratories Inc).

B. Enzyme-Linked Immunosorbent Assay (ELISA)

An enzyme-linked immunosorbent assay, or ELISA, may be used to measure the differential expression of a plurality of biomarkers. There are many variations of an ELISA assay. ELISA tests may be formatted for direct, indirect, competitive, or sandwich detection of the analyte. All are based on the immobilization of an antigen or antibody on a solid surface, generally a microtiter plate. The original ELISA method comprises preparing a sample containing the biomarker proteins of interest, coating the wells of a microtiter plate with the sample, incubating each well with a primary antibody that recognizes a specific antigen, washing away the unbound antibody, and then detecting the antibody-antigen complexes. The antibody-antibody complexes may be detected directly. The primary antibodies are conjugated to a detection system, such as an enzyme that produces a detectable product. The antibody-antibody complexes may be detected indirectly. For example, the primary antibody is detected by a secondary antibody that is conjugated to a detection system, as described above. The microtiter plate is then scanned and the raw intensity data may be converted into expression values using means known in the art. Single- and Multi-probe kits are available from commercial suppliers, e.g., Meso Scale Discovery (MSD).

In one ELISA method, a first, or capture, binding agent, such as an antibody that specifically binds the biomarker of interest, is immobilized on a suitable solid phase substrate or carrier. The test biological sample is then contacted with the capture antibody and incubated for a desired period of time. After washing to remove unbound material, a second, detection, antibody that binds to a different, non-overlapping, epitope on the biomarker is then used to detect binding of the polypeptide biomarker to the capture antibody. The detection antibody is preferably conjugated, either directly or indirectly, to a detectable moiety. Examples of detectable moieties that can be employed in such methods include, but are not limited to, cheminescent and luminescent agents; fluorophores such as fluorescein, rhodamine and eosin; radioisotopes; colorimetric agents; and enzyme-substrate labels, such as biotin.

In another embodiment, the ELISA is a competitive binding assay, wherein labeled biomarker is used in place of the labeled detection antibody, and the labeled biomarker and any unlabeled biomarker present in the test sample compete for binding to the capture antibody. The amount of biomarker bound to the capture antibody can be determined based on the proportion of labeled biomarker detected.

In certain embodiments, the biomarker or antibody bound to the biomarker is directly or indirectly labeled with a detectable moiety. The role of a detectable agent is to facilitate the detection step of the diagnostic method by allowing visualization of the complex formed by binding of the binding agent to the protein marker (or fragment thereof). The detectable agent can be selected such that it generates a signal that can be measured and whose intensity is related (preferably proportional) to the amount of protein marker present in the sample being analyzed. Methods for labeling biological molecules such as polypeptides and antibodies are well-known in the art. Any of a wide variety of detectable agents can be used in the practice of the present disclosure. Suitable detectable agents include, but are not limited to: various ligands, radionuclides, fluorescent dyes, chemiluminescent agents, microparticles (such as, for example, quantum dots, nanocrystals, phosphors and the like), photosensitizers, enzymes (such as, those used in an ELISA, i.e., horseradish peroxidase, beta-galactosidase, luciferase, alkaline phosphatase), colorimetric labels, magnetic labels, and biotin, digoxigenin or other haptens and proteins for which antisera or monoclonal antibodies are available.

The antibodies may be attached to imaging agents of use for imaging and diagnosis of various diseased organs, tissues or cell types. The antibody may be labeled or conjugated with a fluorophore or radiotracer for use as an imaging agent. Many appropriate imaging agents are known in the art, as are methods for their attachment to proteins or peptides using metal chelate complexes, radioisotopes, fluorescent markers, or enzymes whose presence can be detected using a colorimetric markers (such as, but not limited to, urease, alkaline phosphatase, (horseradish) hydrogen peroxidase and glucose oxidase). In some embodiments, the imaging conjugate will also be dual labeled with a radio-isotope in order to combine imaging through nuclear approaches and be made into a unique cyclic structure and optimized for binding affinity and pharmacokinetics. Such agents can be administered by any number of methods known to those of ordinary skill in the art including, but not limited to, oral administration, inhalation, subcutaneous (sub-q), intravenous (I.V.), intraperitoneal (I.P.), intramuscular (I.M.), or intrathecal injection, or as described in greater detail below.

In some aspects, the imaging agent is a chromophore, such as a fluorophore. Exemplary fluorophores suitable for use with the present disclosure includes rhodamine, rhodol, fluorescein, thiofluorescein, aminofluorescein, carboxyfluorescein, chlorofluorescein, methylfluorescein, sulfofluorescein, aminorhodol, carboxyrhodol, chlororhodol, methylrhodol, sulforhodol; aminorhodamine, carboxyrhodamine, chlororhodamine, methylrhodamine, sulforhodamine, and thiorhodamine; cyanine, indocarbocyanine, oxacarbocyanine, thiacarbocyanine, merocyanine, cyanine 2, cyanine 3, cyanine 3.5, cyanine 5, cyanine 5.5, cyanine 7, oxadiazole derivatives, pyridyloxazole, nitrobenzoxadiazole, benzoxadiazole, pyren derivatives, cascade blue, oxazine derivatives. Nile red. Nile blue, cresyl violet, oxazine 170, acridine derivatives, pro flavin, acridine orange, acridine yellow, arylmethine derivatives, auramine, crystal violet, malachite green, tetrapyrrole derivatives, porphin, phtalocyanine and bilirubin; 1-dimethylaminonaphthyl-5-sulfonate, 1-anilino-8-naphthalene sulfonate, 2-p-touidinyl-6-naphthalene sulfonate, 3-phenyl-7-isocyanatocoumarin, N-(p-(2-benzoxazolyl)phenyl)maleimide, stilbenes, pyrenes, 6-FAM (Fluorescein), 6-FAM (NHS Ester), Fluorescein dT, HEX, JOE (NHS Ester), MAX, TET, ROX, TAMRA, TARMA™ (NHS Ester), TEX 615, ATTO™ 488, ATTO™ 532, ATTO™ 550, ATTO™ 565, ATTO™ RholOl, ATTO™ 590, ATTO™ 633, ATTO™ 647N, TYE™ 563, TYE™ 665, and TYE™ 705. In particular aspects, the chromophore is TAMRA.

The detectable moiety may include, but is not limited to fluorodeoxyglucose (FDG); 2′-fluoro-2′deoxy-1beta-D-arabinofuranosyl-5-ethyl-uracil (FEAU); 5-[¹²³I]-2′-fluoro-5-iodo-1β-D-arabinofuranosyl-uracil; 5-[¹²⁴I]-2′-fluoro-5-iodo-1β-D-arabinofuranosyl-uracil; 5-[¹³¹I]-2′-fluoro-5-iodo-1β-D-arabinofuranosyl-uracil, 5-[¹⁸F]-2′-fluoro-5-fluoro-1-β-D-arabinofuranosyl-uracil; 2-[¹¹I]- and 5-([¹¹C]-methyl)-2′-fluoro-5-methyl-1-β-D-arabinofuranosyl-uracil; 2-[¹¹C]-2′-fluoro-5-ethyl-1-β-D-arabinofuranosyl-uracil; 5-([¹¹C]-ethyl)-2′-fluoro-5-ethyl-1-β-D-arabinofuranosyl-uracil; 5-(2-[¹⁸F]-ethyl)-2′-fluoro-5-(2-fluoro-ethyl)-1-β-D-arabinofuranosyl-uracil, 5-[¹²³I]-2′-fluoro-5-iodovinyl-1-β-D-arabinofuranosyl-uracil; 5-[¹²⁴I]-2′-fluoro-5-iodovinyl-1-β-D-arabinofuranosyl-uracil; 5-[¹³¹I]-2′-fluoro-5-iodovinyl-1-β-D-arabinofuranosyl-uracil; 5-[¹²³I]-2′-fluoro-5-iodo-1-β-D-ribofuranosyl-uracil; 5-[¹²⁴I]-2′-fluoro-5-iodo-1-β-D-ribofuranosyl-uracil; 5-[¹³¹I]-2′-fluoro-5-iodo-1-β-D-ribofuranosyl-uracil; 5-[¹²³I]-2′-fluoro-5-iodovinyl-1-β-D-ribofuranosyl-uracil; 5-[¹²⁴I]-2′-fluoro-5-iodovinyl-1-β-D-ribofuranosyl-uracil; 5-[¹³¹I]-2′-fluoro-5-iodovinyl-1-β-D-ribofuranosyl-uracil; or 9-4-[¹⁸F]fluoro-3-(hydroxymethyl)butyl]guanine.

In some aspects, the imaging agent is a radionuclide. Suitable radionuclide labels are Tc, In, Ga, Cu, F, Lu, Y Bi, Ac, and other radionuclide isotopes. Particularly, the radionuclide is selected from the group comprising ¹¹¹In, ^{99m}Tc, ^{94m}Tc, ⁶⁷Ga, ⁶⁶Ga, ⁶⁸Ga, ⁵²Fe, ⁶⁹Er, ⁷²As, ⁹⁷Ru, ²³Pb, ⁶²Cu, ⁶⁴Cu, ⁶⁷Cu, ¹⁸⁶Re, ¹⁸⁸Re, ⁸⁶Y, ⁹⁰Y, ⁵¹Cr, ^{52m}Mn, ¹⁵⁷Gd, ¹⁷⁷Lu, ¹⁶¹Tb, ¹⁶⁹Yb, ¹⁷⁵Yb, ¹⁰⁵Rh, ¹⁶⁶Dy, ¹⁶⁶Ho, ¹⁵³Sm, ¹⁴⁹Pm, ¹⁵¹Pm, ¹⁷²Tm, ¹²¹Sn, ^{177m}Sn, ²¹³Bi, ¹⁴²Pr, ¹⁴³Pr, ¹⁹⁸Au, ¹⁹⁹Au, ¹⁸F, ¹²³I, ¹²⁴I, ¹³¹I, ⁷⁵Br, ⁷⁶Br, ⁷⁷Br, and ⁸²Br, amongst others. These radionuclides are cationic and can be complexed with the chelator through the chelating group of the conjugate to form labeled compositions.

Methods of detecting and/or for quantifying a detectable label or signal generating material depend on the nature of the label. The products of reactions catalyzed by appropriate enzymes can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers. Any of the methods for detection can be performed in any format that allows for any suitable preparation, processing, and analysis of the reactions. This can be, for example, in multi-well assay plates (e.g., 96 wells or 386 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label. Imaging may be by optical imaging, ultrasound, PET, SPECT, MRI, or phototherapy.

In certain embodiments, the antigen-specific antibodies may be immobilized on a carrier or support (e.g., a bead, a magnetic particle, a latex particle, a microtiter plate well, a cuvette, or other reaction vessel). Examples of suitable carrier or support materials include agarose, cellulose, nitrocellulose, dextran, Sephadex®, Sepharose®, liposomes, carboxymethyl cellulose, polyacrylamides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, and the like. Binding agents may be indirectly immobilized using second binding agents specific for the first binding agents (e.g., mouse antibodies specific for the protein markers may be immobilized using sheep anti-mouse IgG Fc fragment specific antibody coated on the carrier or support).

In other aspects, the deglycosylated protein may be detected by a multiplex ELISA to detect two or three of the deglycosylated protein simultaneously. For example, the multiplex ELISA may comprise an antibody array with capture antibodies spotted in subarrays on which the sample is incubated, non-specific proteins are washed off, and the array is incubated with a cocktail of biotinylated detection antibodies followed by a streptavidin-conjugated fluorophore which is visualized by a fluorescence laser scanner (e.g., Quantibody Multiplex ELISA Array, RayBiotech).

The presence of several different deglycosylated proteins in a test sample can be detected simultaneously using a multiplex assay, such as a multiplex ELISA. Multiplex assays offer the advantages of high throughput, a small volume of sample being required, and the ability to detect different proteins across a board dynamic range of concentrations. In certain embodiments, such methods employ an array, wherein multiple binding agents (for example, capture antibodies) specific for multiple deglycosylated proteins are immobilized on a substrate, such as a membrane, with each capture antibody being positioned at a specific, pre-determined, location on the substrate. Methods for performing assays employing such arrays include those described, for example, in US Patent Publication Nos. US2010/0093557A1 and US2010/0190656A1, the disclosures of which are hereby specifically incorporated by reference.

Multiplex arrays in several different formats based on the utilization of, for example, flow cytometry, chemiluminescence or electron-chemiluminescence technology, are well known in the art. Flow cytometric multiplex arrays, also known as bead-based multiplex arrays, include the Cytometric Bead Array (CBA) system from BD Biosciences (Bedford, Mass.) and multi-analyte profiling (xMAP®) technology from Luminex Corp. (Austin, Tex.), both of which employ bead sets which are distinguishable by flow cytometry. Each bead set is coated with a specific capture antibody. Fluorescence or streptavidin-labeled detection antibodies bind to specific capture antibody-protein complexes formed on the bead set. Multiple deglycosylated proteins can be recognized and measured by differences in the bead sets, with chromogenic or fluorogenic emissions being detected using flow cytometric analysis.

In an alternative format, a multiplex ELISA from Quansys Biosciences (Logan, Utah) coats multiple specific capture antibodies at multiple spots (one antibody at one spot) in the same well on a 96-well microtiter plate. Chemiluminescence technology is then used to detect multiple deglycosylated proteins at the corresponding spots on the plate.

An antibody microarray may also be used to measure the differential expression of a plurality of deglycosylated proteins. For this, a plurality of antibodies is arrayed and covalently attached to the surface of the microarray or biochip. A protein extract containing the proteins of interest is generally labeled with a fluorescent dye or biotin. The labeled proteins are incubated with the antibody microarray. After washes to remove the unbound proteins, the microarray is scanned. The raw fluorescent intensity data may be converted into expression values using means known in the art.

### III. METHODS OF TREATMENT

Further provided herein are methods for treating or delaying progression of cancer in an individual comprising administering to the individual an effective amount of an anti-cancer therapy, such as an immune checkpoint inhibitor (e.g., described below), to a subject determined to have an increased expression of a protein, such as an immune checkpoint protein (e.g., PD-L1), by the methods provided herein.

The cancer may be breast cancer, lung cancer, prostate cancer, ovarian cancer, brain cancer cell, liver cancer, cervical cancer, colon cancer, renal cancer, skin cancer, head and neck cancer, bone cancer, esophageal cancer, bladder cancer, uterine cancer, lymphatic cancer, stomach cancer, pancreatic cancer, testicular cancer, intestinal cancer, lymphoma, or leukemia. In some embodiments, the subject is a mammal, e.g., a primate, preferably a higher primate, e.g., a human (e.g., a patient having, or at risk of having, a disorder described herein). In addition, other immune checkpoint-associated diseases, such as diabetes, neurodegenerative disease, or rheumatic disease, may be treated with the present methods.

In one embodiment, the subject is in need of enhancing an immune response. In certain embodiments, the subject is, or is at risk of being, immunocompromised. For example, the subject is undergoing or has undergone a chemotherapeutic treatment and/or radiation therapy. Alternatively, or in combination, the subject is, or is at risk of being, immunocompromised as a result of an infection.

A. Pharmaceutical Compositions

Also provided herein are pharmaceutical compositions and formulations comprising an anti-cancer therapy, such as an immune checkpoint inhibitor, and a pharmaceutically acceptable carrier for subjects determined to have an increased expression of a protein, such as an immune checkpoint protein.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes), and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.).

B. Anti-Cancer Therapies

In certain embodiments, the compositions and methods of the present embodiments involve an anti-cancer therapy which may be administered in combination with at least one additional therapy. The anti-cancer therapy and/or additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The therapy may be in the form of adjuvant or neoadjuvant therapy.

In some embodiments, the anti-cancer therapy and/or additional therapy is the administration of small molecule enzymatic inhibitor or anti-metastatic agent. In some embodiments, the anti-cancer therapy and/or additional therapy is the administration of side-effect limiting agents (e.g., agents intended to lessen the occurrence and/or severity of side effects of treatment, such as anti-nausea agents, etc.). In some embodiments, the anti-cancer therapy and/or additional therapy is radiation therapy. In some embodiments, the anti-cancer therapy and/or additional therapy is surgery. In some embodiments, the anti-cancer therapy and/or additional therapy is a combination of radiation therapy and surgery. In some embodiments, the anti-cancer therapy and/or additional therapy is gamma irradiation. In some embodiments, the anti-cancer therapy and/or additional therapy is therapy targeting PBK/AKT/mTOR pathway, HSP90 inhibitor, tubulin inhibitor, apoptosis inhibitor, and/or chemopreventative agent. The anti-cancer therapy and/or additional therapy may be one or more of the chemotherapeutic agents known in the art.

The anti-cancer therapy may be administered before, during, after, or in various combinations relative to an additional cancer therapy, such as immune checkpoint therapy. The administrations may be in intervals ranging from concurrently to minutes to days to weeks. In embodiments where the first therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the anti-cancer therapy and additional therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below a first anti-cancer therapy, such as an immune checkpoint inhibitor, is “A” and an additional anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclophosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azasrine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO) retinoids, such as retinoic acid; capecitabine; carboplatin, procarbazine, plicomycin, gemcitabien, navelbine, farnesyl-protein transferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation, and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-rug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment. As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting. i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies include immune adjuvants, e.g., Mycobacterium bovis, Plasmodium falciparum, dinitrochlorobenzene, and aromatic compounds; cytokine therapy. e.g., interferons α, β, and γ, IL-1, GM-CSF, and TNF; gene therapy, e.g., TNF, IL-1, IL-2, and p53; and monoclonal antibodies, e.g., anti-CD20, anti-ganglioside GM2, and anti-p185. It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., Pardoll, 2012; incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present invention. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PD-L1 and/or PD-L2. In another embodiment, a PD-L1 binding antagonist is a molecule that inhibits the binding of PD-L1 to its binding partners. In a specific aspect, PD-L1 binding partners are PD-1 and/or B7-1. In another embodiment, the PD-L2 binding antagonist is a molecule that inhibits the binding of PD-L2 to its binding partners. In a specific aspect, a PD-L2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Publication Nos. US20140294898 and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475 Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in U.S. Pat. No. 8,119,129 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application Nos. WO2001014424, and WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mobs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### IV. KIT

Also within the scope of the present disclosure are kits for detecting glycosylated, such as those disclosed herein. An example of such a kit may include a deglycosylation enzyme and one or more antibodies. The kit may further comprise instructions for use of the antibodies to detect the presence or absence of the specific glycosylated proteins, such as immune checkpoint proteins, described herein. The kit may further comprise instructions for diagnostic purposes, indicating that a positive identification of immune checkpoint proteins described herein in a sample from a cancer patient indicates sensitivity to the immune checkpoint inhibitor. The kit may further comprise instructions that indicate that a positive identification of immune checkpoint proteins described herein in a sample from a cancer patient indicates that a patient should be treated with an immune checkpoint inhibitor.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—PD-L1 Detection Method

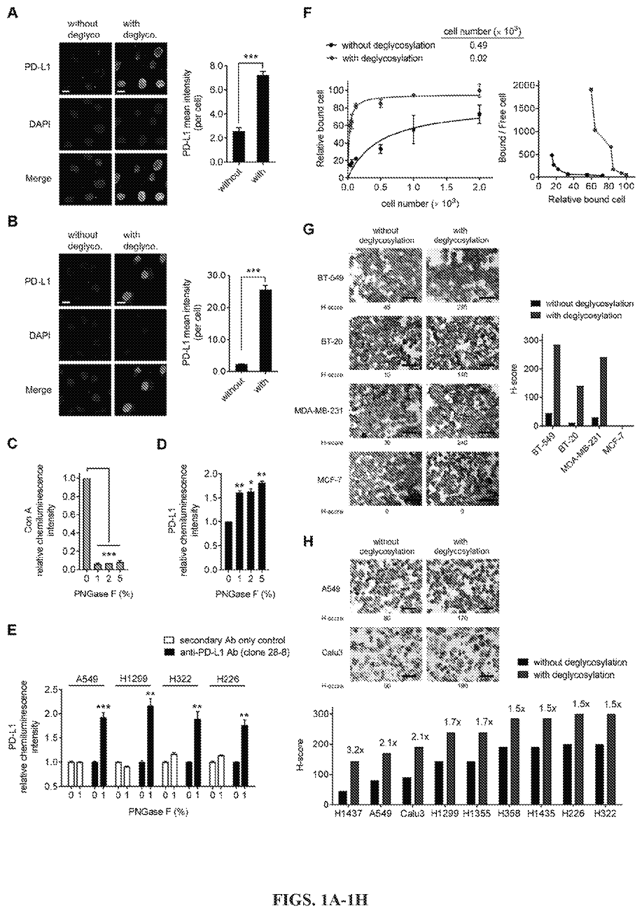

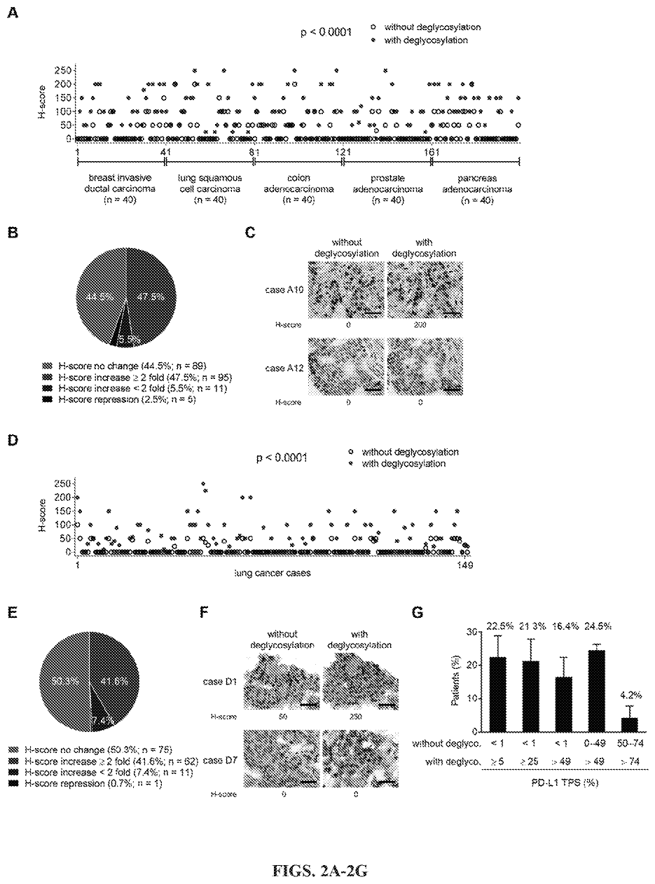

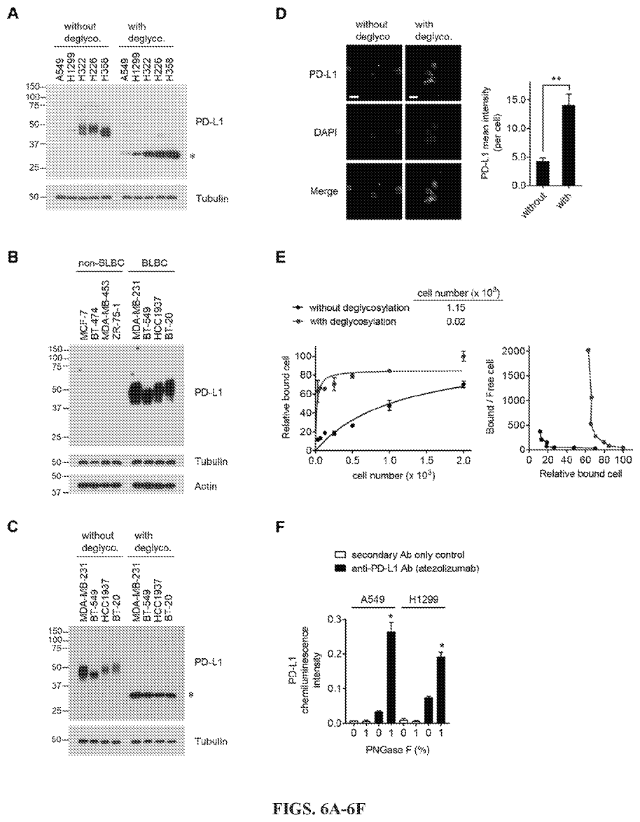

Removal of N-linked glycosylation enhances anti-PD-L1 signal in human cancer cells: The migration pattern of PD-L1 on gel electrophoresis was heterogeneous as illustrated by a range of bands at ˜50 kDa with heavy glycosylation in a panel of human lung and basal-like breast cancer (BLBC) but not non-BLBC cell lines (FIGS. 6A and 6B). Treatment with recombinant glycosidase (peptide-N-glycosidase F; PNGase F) to remove the entire N-linked glycosylation (deglycosylation, herein after) resulted in a homogenous pattern of PD-L1 at ˜33 kDa (FIGS. 6A and 6C). To determine whether the N-linked glycan structure of PD-L1 hinders antibody-based detection targeting the PD-L1 antigen, cells were first pretreated with or without PNGase F followed by immunofluorescence confocal microscopy analysis. The fluorescent intensity of PD-L1 was significantly enhanced after PNGase F treatment in lung cancer and BT-549 BLBC cells compared with no treatment (FIGS. 1A, 1B, and 6D). The results were further supported by an enzyme-linked immunosorbent assay (ELISA)-based method. First, concanavalin A (Con A), a lectin representing a glycoprotein precursor, was used as a positive control to quantitatively measure the deglycosylation efficiency of PNGase F, and substantially reduced chemiluminescence intensity of Con A was observed (FIG. 1C). Following PNGase F treatment, anti-PD-L signal was significantly increased in BT-549 BLBC cells (FIG. 1D) as determined by the FDA approved diagnostic rabbit monoclonal antibody (clone 28-8 mAb) against the extracellular domain of human PD-L1 (Phillips et al., 2015). Similar results were observed in other lung cancer cells (FIG. 1E). Next, the dissociation constant (Kd) of the binding of clone 28-8 mAb to PD-L1 was determined, which revealed a ˜25- and 55-fold increase in binding affinity after deglycosylation in A549 and H1299 cells, respectively (FIGS. 1F and 6E). It is worth noting that in addition to the improved PD-L1 detection, anti-PD-L1 signal detected by another FDA approved therapeutic PD-L1 antibody, atezolizumab, was also significantly enhanced after deglycosylation in lung cancer cells (FIG. 6F). Similar results demonstrated by different PD-L1 antibodies further supported the notion that glycans on the PD-L1 antigen region hinders its interaction with and subsequent detection by PD-L1 antibodies.

Deglycosylation enhances PD-L1 detection in tumor tissue blocks by IHC staining: PD-L1 IHC assay is the standard method used in the clinic to stratify patients for immune checkpoint therapy. To address whether sample deglycosylation is suitable for PD-L1 expression assessment by IHC, formalin-fixed paraffin-embedded (FFPE) tissue blocks of cancer cell lines were first utilized as a test model and to examine the effects of deglycosylation on anti-PD-L1 signals. Consistently, PD-L1 expression levels as determined by histoscore (H-score) were enhanced after glycan removal in lung cancer and BLBC cells, but not in the MCF-7 cells that do not express PD-L1 (FIGS. 1G and 1H). Together, these results indicated that N-linked glycosylation of PD-L1 impedes its detection by PD-L1 antibodies, and the removal of PD-L1 glycan structure likely eliminates the steric hindrance for antibody recognition, which can significantly improve the antibody-based detection sensitivity.

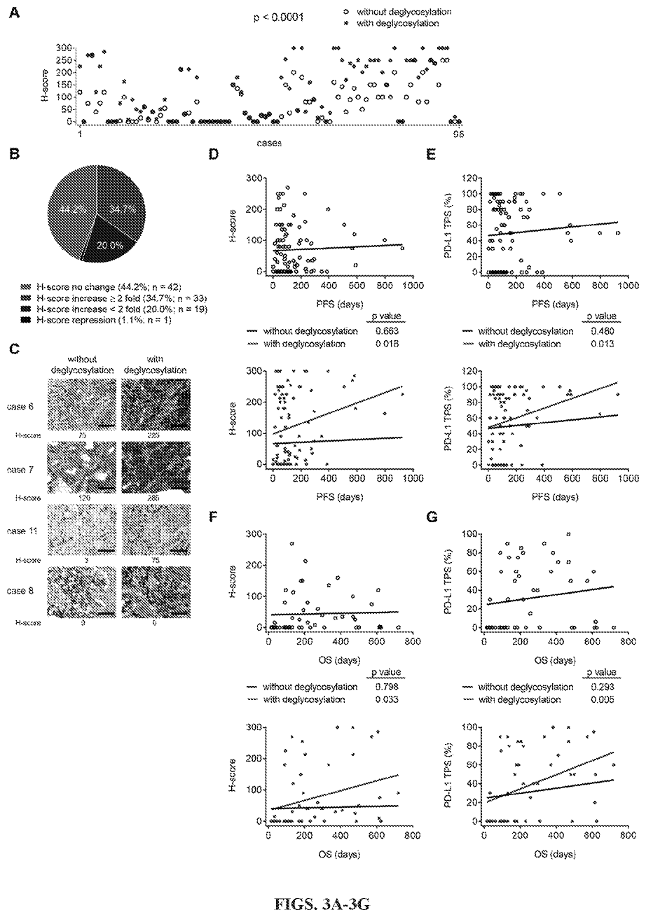

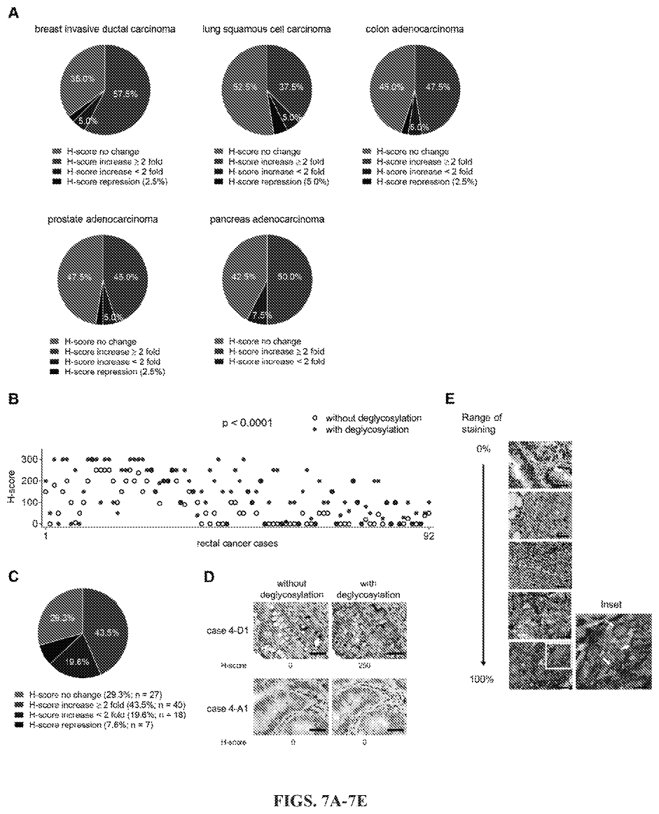

Deglycosylation significantly enhances anti-PD-L1 signal in human tumor tissue microarray: The anti-PD-L1 signals were further evaluated in pathological staining of patient samples from a multi-organ carcinoma tissue microarray (TMA) that included five cancer types: breast, lung, colon, prostate, and pancreatic cancers (n=200; FIGS. 2A-2C). The H-score representing PD-L1 protein expression between samples processed with and without deglycosylation varied significantly (p<0.0001; FIG. 2A). Among 200 cases analyzed, the differences in H-score with and without glycosylation removal were categorized into two major groups: patients whose H-scores did not change (44.5%; n=89) and those that increased by more than two folds (47.5%; n=95) (FIG. 2B; representative images shown in FIG. 2C). A similar pattern was observed in individual analysis of five cancer cohorts in a multi-organ carcinoma TMA (FIG. 7A) and in two independent patient cohorts in lung (n=149; FIGS. 2D-2F) and rectal (n=92; FIGS. 71-7D) cancers. These results revealed that the number of patients (37.5-57.5%) with positive IHC staining for PD-L1 increased significantly by more than two folds after deglycosylation, indicating N-linked glycosylation of PD-L1 critically affects its recognition by the PD-L1 antibody in clinical diagnosis of various cancer types. tumor tissues were further analyzed from three independent cohorts of lung cancer patients in which PD-L1 tumor proportion score (TPS) defined by the percentage of PD-L1 positive cells in tumor cells was detected at <1% or within 0-49% by conventional IHC without prior deglycosylation. Among them, sample deglycosylation significantly increased PD-L1 TPS to ≥5% and >49%, the clinically agreed-upon cutoffs to be considered eligible for nivolumab and pembrolizumab therapy, respectively. On the basis of those findings without consideration for response rates (FIGS. 2G and 7E), the removal of N-linked glycosylation identified about 16.4-24.5% of patient population who could have benefited from anti-PD-1/PD-L1 therapy but were excluded based on the current staining method. Interestingly, sample deglycosylation increased PD-L1 expression only in a relatively small population (4.2%) of patients whose PD-L1-positive cells were >49% by conventional IHC without deglycosylation (FIG. 2G). Together, the proposed sample deglycosylation may be a feasible method to eliminate or reduce false-negative PD-L1 expression and has the potential to benefit a significant population of patients with false-negative PD-L1 detection (within 0-49% by conventional IHC staining), rendering them eligible for immune checkpoint therapy after glycan removal.

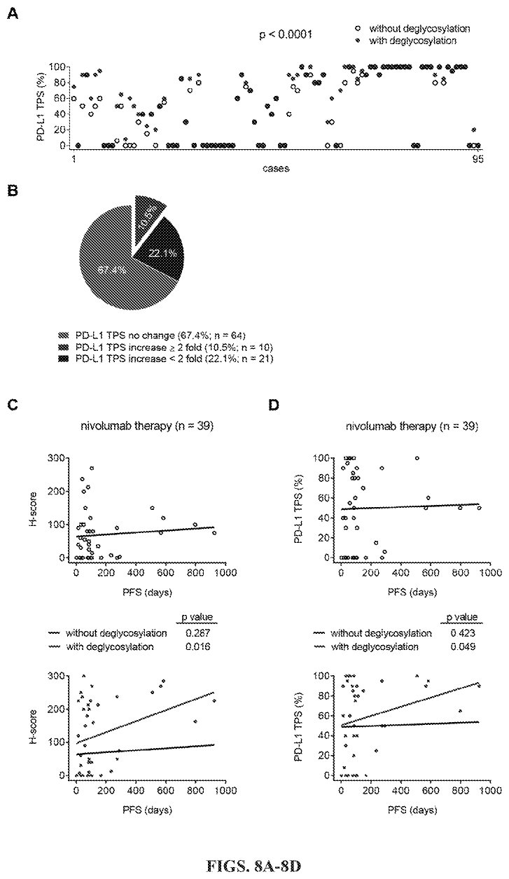

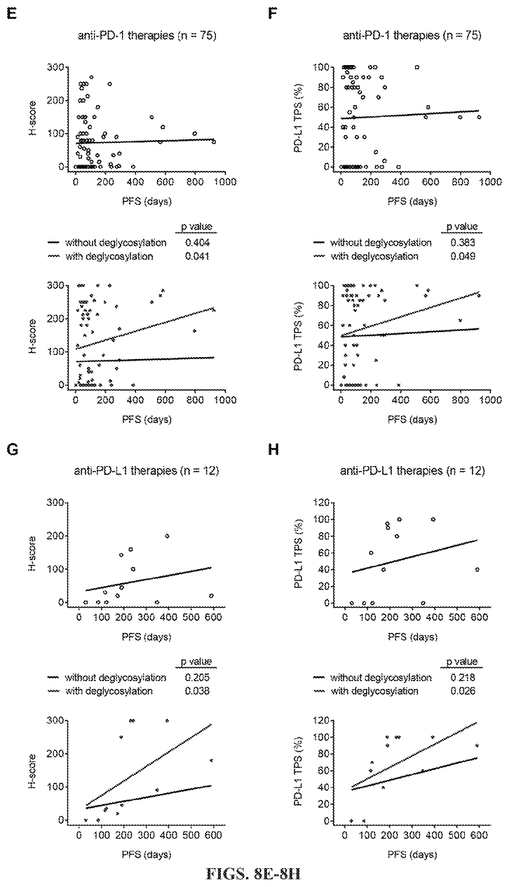

Improved PD-L1 detection after deglycosylation is associated with response to anti-PD-1/PD-L1 therapy To address the inconsistent observations between PD-L1 IHC readout and patient response, which has been a long-term puzzle in the clinic, 95 pre-immunotherapy archived FFPE blocks were collected containing tumor tissues from patients with different types of cancers who received or were undergoing immunotherapy. Identical blocks were treated with or without PNGase F glycosidase and subjected to IHC staining followed by correlation analysis between pathological PD-L1 expression and clinical response rates. Consistently, the H-score of samples processed with deglycosylation increased significantly compared with those without deglycosylation (p<0.0001; FIG. 3A). The fold changes in H-score were further grouped after deglycosylation into four categories: 1) no change (44.2%), 2) increased by more than twofold (34.7%), 3) increased by less than twofold (20%), and 4) repression within two folds (1.1%) (FIG. 3B; representative images in FIG. 3C). In addition, the percentage of PD-L1-positive signal also varied significantly between samples processed with and without deglycosylation (p<0.0001; FIG. 8A). Two comparable groups were identified: a) patients whose PD-L1 TPS did not change (67.4%) and b) those that increased (32.6%) among which 10.5% (n=10) increased by more than twofold (FIG. 8B). Notably, the H-score readout of PD-L1 correlated significantly with the patient progression-free survival (PFS) only after sample pretreatment with PNGase F but not without PNGase F (FIG. 3D; p=0.018 versus p=0.663). The improved p value was also observed in the correlation between the PD-L1 TPS and the patient PFS in the presence of PNGase F (FIG. 3E; p=0.013 versus p=0.480). Statistical analyses of pathological PD-L1 levels and PFS of the majority of patients in the cohort, who received anti-PD-1 therapy nivolumab (FIGS. 8C and 8D; n=39), showed improved p values between the PFS following nivolumab therapy and the PD-L1 H-score readout (FIG. 8C; p=0.016 versus p=0.287) or the PD-L1 TPS (FIG. 8D; p=0.049 versus p=0.423) in the presence of PNGase F. In addition, similar results were also observed in other groups of patients who received anti-PD-1 therapy, e.g., nivolumab, pembrolizumab, and camrelizumab (FIGS. 8E and 8F; n=75), or anti-PD-L1 therapy. e.g., atezolizumab and durvalumab (FIGS. 8G and 8H; n=12), in the same cohort. Together, sample deglycosylation renders a more accurate assessment of PD-L1 levels to predict clinical outcomes of patients. In addition to the PFS, 49 cases of this cohort were identified with available overall survival (OS) to study the correlation between the OS of patients and pathological PD-L1 levels. The results indicated similarly improved p values for the correlation between the OS and PD-L1 H-score readout (FIG. 3F; p=0.033 versus p=0.798) or PD-L1 TPS (FIG. 3G; p=0.005 versus p=0.293) after PNGase F treatment. Collectively, using both PFS and OS, it was demonstrated that sample deglycosylation indeed resulted in a more accurate assessment of PD-L1 expression, allowing better prediction of clinical response to anti-PD-1/PD-L1 therapy.

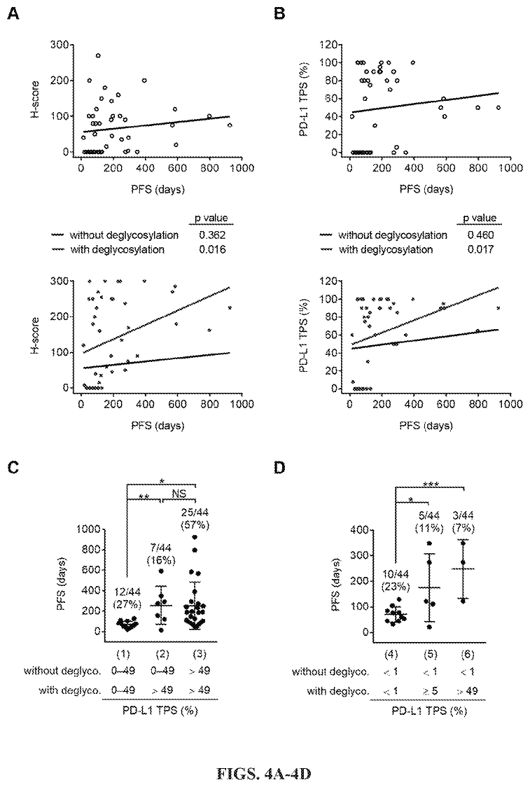

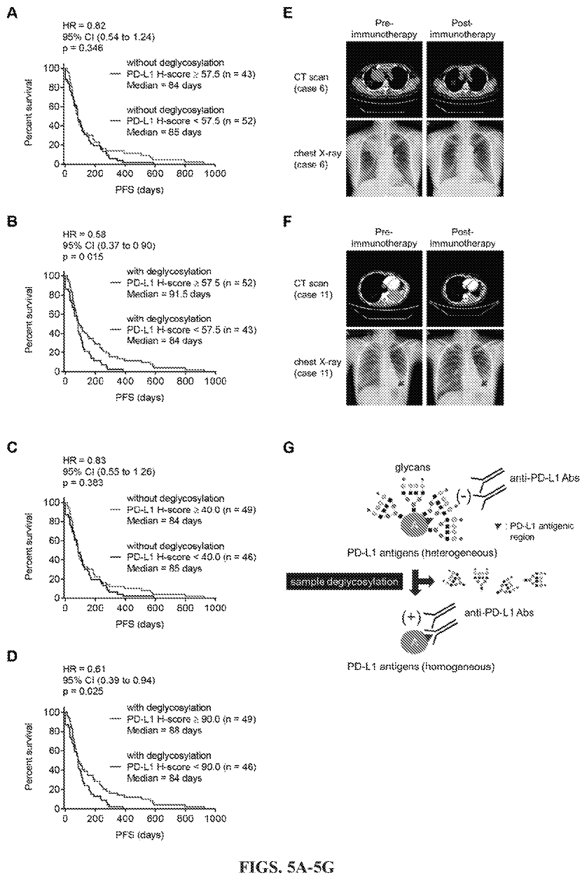

Increased PD-L1 signal after deglycosylation is beneficial to therapeutic selection: Traditionally in lung cancer, patients whose PD-L1 expression is <1% are excluded from anti-PD-1/PD-L1 therapy whereas those with ≥1% are preferentially administered immunotherapy alone (>49%) or with concurrent chemotherapy (1-49%). Among those 95 cases, it was found that deglycosylation of tissue samples from a major group of lung cancer patients (n=44) also significantly improved the correlation between the patient PFS and the pathological PD-L1 levels determined either by PD-L1 H-score (FIG. 4A; p=0.016 versus p=0.362) or PD-L1 TPS (FIG. 4B; p=0.017 versus p=0.460). Next, it was asked whether patients within the 0-49% PD-L1 TPS by conventional IHC would benefit from sample deglycosylation in therapeutic selection to increase PD-L1-positive cells to >49%. A significant increase in the PFS was observed in group 2 compared with group 1 (FIG. 4C; p=0.003; mean, 256.6 days versus 70.1 days), suggesting that about 16% of patients in group 2 whose PD-L1 TPS appeared to be detected inaccurately by conventional IHC would therapeutically benefit from sample deglycosylation to increase PD-L1 TPS to >49%. Notably, the PFS of patients in group 2 was comparable to those in group 3 whose PD-L1 TPS were >49% with and without deglycosylation (FIG. 4C; mean, 256.6 days versus 252.9 days), indicating that PD-L1 levels after deglycosylation more accurately predicts clinical outcomes. Thus, the deglycosylation-mediated increase in PD-L1 signal could render 16% of patients (group 2; FIG. 4C) for immunotherapy alone instead with concurrent chemotherapy.

Next, to further investigate whether patients whose PD-L1 expression is <1% would benefit from sample deglycosylation by increasing PD-L1 TPS to greater than 5% (FIG. 4D; designated as group 5) or >49% (FIG. 4D; group 6), the PFS of lung cancer patients (15 out of 44) with <1% PD-L1 TPS was analyzed by conventional IHC. A significant increase in the PFS was observed between group 4 and group 5 (p=0.029; mean, 70.9 days versus 175.2 days) and between group 4 and group 6 (p=0.0006; mean, 70.9 days versus 248.0 days). This suggested that about 7-11% of patient population in this cohort, whose PD-L1 expression was <1% staining by conventional IHC, increased to >49% staining (7% of patient population) or ≥5% staining (11% of patient population) after sample deglycosylation, and those patients appeared to respond to anti-PD-1/PD-L1 therapy (FIG. 4D), but would have otherwise been ineligible for the immune checkpoint therapy. Indeed, this number is close to the estimated potential PD-L1 false-negative patient population (9-17%) who still responded to immunotherapy in clinical trials. Collectively, sample deglycosylation identified a significant population (7-16%) of patients who are eligible to receive immune checkpoint inhibitors and likely benefit from the treatment, especially those with false-negative detection of PD-L1 within 0-49% by conventional IHC staining.

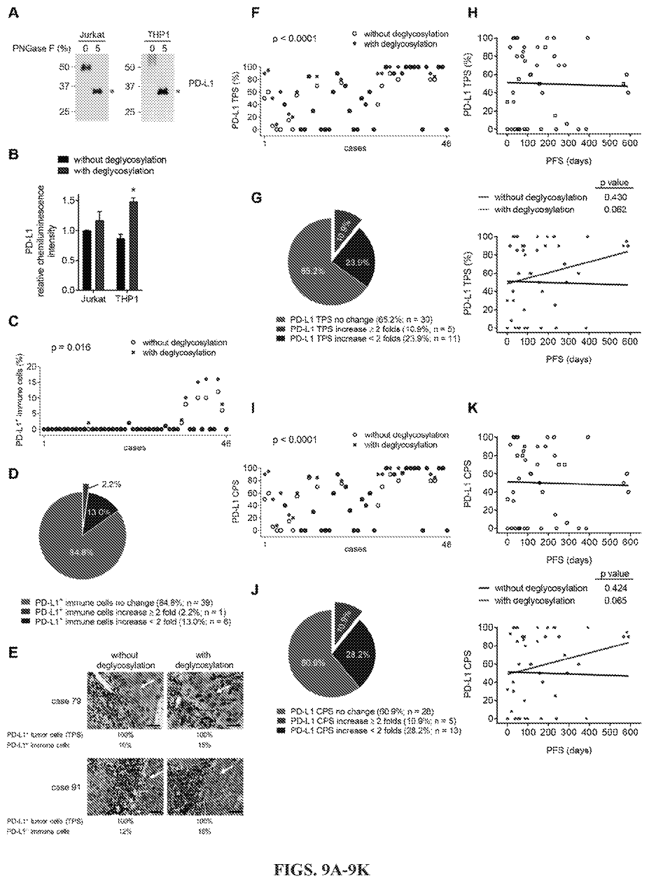

PD-L1 deglycosylation enhances its detection in a small fraction of tumor-associated immune cells: PD-L1 expression score in immune and tumor cells has been assessed in patients who received PD-L1 inhibitors, such as atezolizumab (Fehrenbacher et al., 2016; Kowanetz et al., 2018). To determine whether deglycosylation also affects the detection of PD-L1 in immune cells, the status of PD-L1 glycosylation was first assessed in human immune cells, e.g., Jurkat (T lymphocytes) and THP1 (monocytes), by immunoblotting. Pretreatment with PNGase F resulted in a homogenous pattern of PD-L1 at ˜33 kDa in both Jurkat and THP1 cells (asterisks; nonglycosylated PD-L1; FIG. 9A), indicating heavy glycosylation of PD-L1 also occurs in human immune cells. A quantitative ELISA was further performed to determine whether anti-PD-L1 signal intensity is affected after N-linked glycosylation removal in Jurkat and THP1 cells. Following PNGase F treatment, anti-PD-L1 signal intensity was significantly enhanced in THP1 cells but only slightly increased in Jurkat cells (FIG. 9B). These results suggested that the degree of increase in the intensity of anti-PD-L1 signal in immune cells after deglycosylation may vary in different types of immune cells.

Next, the effects of deglycosylation were validated on PD-L1 detection in tumor-associated immune cells from the existing clinical samples in FFPE tissue blocks. Due to the presence of immune cell infiltration in the tumor microenvironment, about 46 out of 95 cases containing tumor-associated immune cells (lymphocytes) were available for reassessment (FIGS. 9C-9E). The percentage of PD-L1-positive signal in immune cells (% PD-L1+immune cells) varied between samples processed with and without deglycosylation but was less significant compared with tumor cells (p=0.016 versus p<0.0001; FIG. 9C versus FIG. 9F).

Moreover, the distribution of increase in PD-L1 detection after deglycosylation in immune cells was proportionally less than that in tumor cells. Specifically, the increase in the percentage of PD-L1+immune cells (FIG. 9D; 15.2%) was less than that in tumor cells (FIG. 9G; 34.8%). Likewise, the percentage that increased by more than twofold were only 2.2% compared with that of 10.9% in tumor cells after deglycosylation (FIG. 9D versus FIG. 9G). Notably, the deglycosylation-mediated increase in PD-L1 intensity change and clinical outcome of the TPS of this cohort (FIGS. 9F-9H) was similar to that of the combined positive score (CPS) (FIGS. 9I-9K), in which PD-L1 was scored in both tumor and immune cells (Kulangara et al., 2019), supporting the minimal effects of deglycosylation of PD-L1 on scoring immune cell PD-L1 expression. In brief, both TPS and CPS of PD-L1 varied significantly between samples processed with and without deglycosylation (p<0.0001; FIGS. 9F and 9I). The distribution of increase in PD-L1 TPS and CPS after deglycosylation was also proportionally comparable (FIGS. 9G and 9J). Moreover, in the presence of PNGase F, the correlation between patient response and either PD-L1 TPS (FIG. 9H; p=0.062 versus p=0.430) or CPS (FIG. 9K; p=0.065 versus p=0.424) demonstrated a near-significant trend. These results suggested that measuring PD-L1 levels either by TPS or CPS following deglycosylation more accurately predicts anti-PD-1/PD-L1 clinical outcome.

In summary, sample deglycosylation of the current cohort enhanced the detection of PD-L1 in a small fraction of tumor-associated immune cells (lymphocytes). In addition, the increase in the number of positive-responding cells by more than twofold was less significant in immune cells than in tumor cells (2.2% versus 10.9%), implying that the profiles of glycan composition between these two cell types may be different. The data indicated that the improvement in PD-L1⁺ immune cells detection was statistically significant (FIG. 9C; p=0.016). Interestingly, statistical significance of the effects of the patient PFS correlation was not observed on scoring PD-L1 TPS or CPS in this cohort (FIGS. 9H and 9K), suggesting that protein deglycosylation might improve PD-L1 scoring in immune cells in a certain population of patients.

Antigen retrieval by protein deglycosylation improves predictive ability of PD-L1 as a biomarker for immunotherapy: Finally, to study whether deglycosylated PD-L1 in tumor cells increases the predictive power of PD-L1 as biomarker to guide anti-PD-1/PD-L1 therapy in clinical practice, PD-L1 H-score values were divided into high or low using the median value from a total 95 cases in both groups treated with and without PNGase F as a cutoff (H-score=57.5). No statistically significant benefits in the PFS of patients with high levels of PD-L1 (FIG. 5A; p=0.346) was observed by conventional IHC, which is consistent with results from multiple clinical trials.

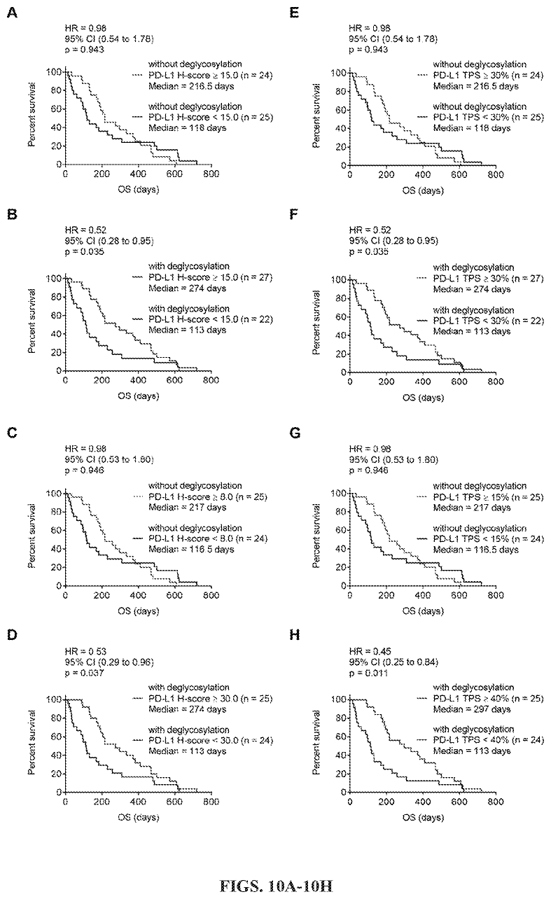

However, with deglycosylation by pretreating samples on IHC slides with PNGase F, patients with high levels of PD-L1 exhibited significantly improved response to immunotherapy that associated with a decline in an estimated hazard ratio (HR) from 0.82 to 0.58 (FIGS. 5A and 5B) compared with those with low levels of PD-L1 (FIG. 5B; p=0.015). Similar results were observed using the respective median value of PD-L1 H-score as a cutoff in the groups treated either with or without PNGase F (FIGS. 5C and 5D). Together with the results from OS analysis using the median value of PD-L1 H-score (FIGS. 10A-10D) or PD-L1 TPS (FIGS. 10E-10H) as a cutoff, the removal of N-linked glycosylation enhances the predictive power of PD-L1 as a biomarker to guide immunotherapy.

The clinical response was also validated by lung imaging screening which demonstrated an increase in H-score by greater than twofold in three randomly selected cases (cases 6, 7, and 11) after deglycosylation. Interestingly, tumors from 2 out of 3 patients exhibited apparent shrinkage under PD-1 inhibitor treatment (arrows; case 6 in FIGS. 3C and 5E; case 11 in FIGS. 3C and 5F), which further illustrated the objective to identify the most responsive patient group.

Collectively, these results suggested that removing the glycan moieties from tumor samples prior to IHC staining leads to a more accurate assessment of PD-L1 expression to allow better prediction of clinical response to anti-PD-1/PD-L1 therapy. On the basis of the current findings, a model (FIG. 5G) is presented showing that heavy glycosylation of PD-L1 hinders its detection by PD-L antibodies which could lead to inaccurate readout from a variety of bioassays, such as HC, ELSA, immunofluorescence microscopy, and immunoblotting. Here, it was demonstrated that removal of PD-L1 N-linked glycosylation from tissue samples by enzymatic digestion increases the homogeneity of target proteins and more precisely antibody-based PD-L1 detection to prevent false-negative readouts. Therefore, deglycosylation of PD-L1 prior to detection may be a more accurate method to quantify its expression than conventional IHC to identify patients who may receive the most benefit from immune checkpoint therapy.

### Example 2—Materials and Methods

Cell culture: All human cells lines cultured at 37° C. under 5% CO2 were obtained from the American Type Culture Collection (Manassas, Va., USA), including breast cancer (BT-549, BT-20, MDA-MB-231, MCF-7), lung cancer (H1437, A549, Calu3, H1299, H1355, H358, H1435, H226, H322), and immune (Jurkat T lymphocytes, THP1 monocytes) cell lines. Human breast cancer cell lines (BT-549, BT-20, MDA-MB-231, MCF-7) and H1435 cells are female-derived cell lines; other cell lines used are male-derived cells. All cell lines were independently validated by STR DNA fingerprinting at The University of Texas MD Anderson Cancer Center and characterized as mycoplasma negative. BT-549, BT-20, MDA-MB-231, MCF-7, and A549 cells were maintained in Dulbecco's modified Eagle's medium (DMEM)/F12, supplemented with 10% fetal bovine serum (FBS) and 1% antibiotic mixture. Calu3 cells were cultured in Eagle's Minimum Essential Medium, supplemented with 10% FBS and 1% antibiotic mixture. Other cells used were cultured in RPMI-1640, supplemented with 10% FBS and 1% antibiotic mixture.

Human tissue samples: Human tissue samples were collected following the guidelines approved by the Institutional Review Board at China Medical University Hospital (CMUH106-REC1-145), Chang Gung Memorial Hospital (201800036B0), The Affiliated Tumor Hospital of Harbin Medical University, and The University of Texas MD Anderson Cancer Center (LAB05-0127). Written informed consent to publish identifiable images was obtained from patients in all cases at the time of tissue sample collection. All tissue samples were collected before immunotherapy. All clinical information validated our results without selection bias. A total of 95 human tissue samples were obtained from patients with cancers of lung (n=44), head and neck (n=22), esophageal (n=13), bladder (n=5), and others (n=13) (gender: 68 males and 27 females; mean±SD age, 59.29±11.18 years; median age, 59.00 years; range, 25-92 years). Progression free survival (PFS) was obtained from all 95 patients with overall survival (OS) available for 49 patients. The differences in PFS (p=0.395) or OS (p=0.639) between males and females were not significant as determined Student's t test. Pearson correlation test was utilized to confirm an insignificant association of patient age with PD-L1 H-score without deglycosylation (p=0.26) and with deglycosylation (p=0.42). The objective response rate (ORR) and the disease control rate (DCR) (n=93 out of 95 of this cohort with immunotherapy response rate available) were 10.8% and 39.8%, respectively, which are comparable to that reported in clinical trial studies in unselected patients with 14-23% of ORR and 36% of DCR (Califano et al., 2018; Huang et al., 2016; Shukuya and Carbone, 2016).

For the human tumor tissue microarrays (TMAs), the study from 92 cases of rectal cancer was approved by the Institutional Review Board at China Medical University Hospital (CMUH106-REC1-145). Informed consent was obtained from all patients (gender: 66 males and 26 females; mean±SD age, 59.43±12.99 years median age, 59 years; range, 31-90 years). Pearson correlation test was further utilized to confirm an insignificant association of patient age with PD-L1 H-score without deglycosylation (p=0.84) and with deglycosylation (p=0.39). Both human carcinoma TMAs of multi-organ and lung were purchased from Biomax, #BC000119 (n=200) and #NSC151 (n=149), respectively. For the study using different cutoffs as threshold, the mean value was measured from three independent cohorts of lung cancer patients expressing PD-L1 (233 cases total), including a group of 44 out of 95 cancer patients who received immunotherapy, 40 out of 200 cases in the multi-organ cancer TMA, and 149 cases in the lung cancer TMA. Among them, a population of patients was further analyzed in which PD-L1 expression was detected at less than 1% by conventional IHC without deglycosylation. Clinically defined cutoffs of PD-L1 positive cells were then set up, including ≥5%, ≥25%, >49%, and >74%, for those patient samples after deglycosylation.

Detection of deglycosylation in cell lysates by immunoblotting (IB): Cells were lysed in RIPA buffer (150 mM NaCl, 50 mM Tris [pH 7.5], 1% Nonidet P40, and protease inhibitor mixture) and sonicated using a Vibra-Cell sonicator. Following the manufacturer's instruction with a slight modification for PNGase F (NEB Inc., P0704) treatment, 5-20 μg of cell lysates were combined with Glycoprotein Denaturing Buffer and water to make up a 10-μl total reaction volume. The mixture was denatured by heating at 100° C. for 10 min and chilled on ice, and 2 μl of 10×GlycoBuffer 2, 2 μl of 10% Nonidet P40, and 6 μl of water were then added to make up a 20 μl total reaction volume. The denatured mixture was incubated at 37° C. overnight without or with 1 μl of PNGase F to keep the final glycerol concentration equal to 5% and subjected to IB analysis with the indicated antibodies.

Detecting deglycosylation by immunofluorescence confocal microscopy: Cells seeded in 8-well chamber slide were fixed in 4% paraformaldehyde at 4° C. overnight. After washing three times with PBS, the fixed cells were incubated with 1× glycoprotein denaturing buffer (0.5% SDS and 40 mM DTT), denatured by heating at 100° C. for 10 min, and chilled on ice. The denaturing buffer was removed from the chamber, and cells were washed with PBS three times, treated without or with PNGase F (5%) containing PBS at 37° C. overnight, and then subjected to immunofluorescence confocal microscopy. In brief, cells were then permeabilized with 0.5% Triton X-100 for 15 min and blocked with 5% bovine serum albumin (BSA) for 1 hr at room temperature. After the incubation with PD-L1 antibody (1:100; Abcam, ab58810) at 4° C. overnight, cells were incubated with an anti-rabbit secondary antibody tagged with fluorescein isothiocyanate (1:500) at room temperature for 1 hr. Nuclei were stained with DAPI contained in the mounting reagent. Confocal fluorescence images were captured using a Zeiss LSM 710 laser microscope. In all cases, optical sections were obtained through the middle planes of the nuclei, as determined with use of nuclear counterstaining.

Sample deglycosylation in quantitative ELISA-based method: Cells seeded at 1×10³ cells/well in ELISA 96-well plates were fixed in 4% paraformaldehyde at 4° C. overnight. After washing three times with PBS, the fixed cells were incubated with 1× glycoprotein denaturing buffer, denatured by heating at 100° C. for 10 min, and chilled on ice. The denaturing buffer was then removed from the well, washed with PBS three times, treated without or with PNGase F containing PBS at 37° C. overnight, followed by quantitative ELISA-based method. For the detection of PD-L1 or Con A (positive control) in BT-549 cells, cells were pretreated increasing amounts of PNGase F (1, 2, 5%) for comparison with cells without PNGase F (0%). After incubation at 37° C. overnight, the PNGase F-pretreated cells were then blocked with 1% BSA solution at 37° C. for 3 hr. After rinsing three times with PBS with 0.05% Tween 20 (PBST), cells were incubated with an anti-PD-L1 antibody (1:100 in blocking buffer; clone 28-8 mAb) at 4° C. overnight or with HRP-conjugated Con A (1:100 in blocking buffer) at room temperature for 2 hr. Cells were then washed with PBST three times with shaking for 1 min and incubated with a Peroxidase-AffiniPure goat anti-rabbit IgG secondary antibody (1:5,000 in blocking buffer) at room temperature for 1 hr (except for the Con A set). Cells were washed with PBST three more times with shaking, and peroxidase substrate TMB (3,3′,5,5′-tetramethylbenzidine) was added and incubated for 30 min at room temperature. The reaction was terminated by the addition of STOP solution. The optical density representing the chemiluminescence intensity was determined at 450 nm using a BioTek Synergy Neo multi-mode reader and corrected by subtraction of readings at 570 nm. For PD-L1 detection in lung cancer cells, PNGase F-pretreated cells (1% PNGase F) were incubated overnight at 37° C. overnight followed by blocking with 1% BSA solution at 37° C. for 3 hr. After rinsing three times with PBST, cells were incubated with or without (secondary Ab only control) an anti-PD-L1 antibody (1:100 in blocking buffer for clone 28-8 mAb; 1:500 in blocking buffer for atezolizumab) at 4° C. overnight. Cells were then washed with PBST three times with shaking for 1 min and incubated with a Peroxidase-AffiniPure goat anti-rabbit IgG (for clone 28-8 mAb; 1:5000 in blocking buffer) or anti-human IgG (for atezolizumab; 1:5,000 in blocking buffer) secondary antibody at room temperature for 1 hr. Cells were washed with PBST three more times with shaking, and TMB as a peroxidase substrate was added and incubated for 30 min at room temperature. The reaction was terminated by the addition of STOP solution. The optical density representing the chemiluminescence intensity was determined at 450 nm using a BioTek Synergy Neo multi-mode reader and corrected by subtraction of readings at 570 nm.

Detection of PD-L1 antibody binding affinity in quantitative ELISA-based method: A saturation binding assay was performed based on the above-mentioned ELISA-based quantitation to determine the binding affinity of anti-PD-L1 clone 28-8 mAb to cell surface PD-L1 antigen. To calculate the number of cells with antigen sites half-saturated by clone 28-8 mAb, cells were seeded in ELISA 96-well plates at a series of cell numbers (2, 1, 0.5, 0.25, 0.125, 0.0625, and 0.03125×10³ cells/well) and fixed in 4% paraformaldehyde at 4° C. overnight. After washing three times with PBS, the fixed cells were incubated with 1% glycoprotein denaturing buffer, denatured by heating at 100° C. for 10 min, and chilled on ice. The denaturing buffer was then removed from the well, washed with PBS three times, and treated with (1%) or without (0%) PNGase F at 37° C. overnight. The PNGase F-pretreated cells were then blocked with 1% BSA solution at 37° C. for 3 hr. After rinsing three times with PBST, cells were incubated with an anti-PD-L1 antibody (1:100 in blocking buffer; clone 28-8 mAb) at 4° C. overnight. Cells were then washed with PBST three times with shaking for 1 min and incubated with a Peroxidase-AffiniPure goat anti-rabbit IgG secondary antibody (1:5,000 in blocking buffer) at room temperature for 1 hr. Cells were washed with PBST three more times with shaking, and TMB as a peroxidase substrate was added and incubated for 30 min at room temperature. The reaction was terminated by the addition of STOP solution. The optical density representing the chemiluminescence intensity was determined at 450 nm using a BioTek Synergy Neo multi-mode reader and corrected by subtraction of readings at 570 nm. The cell number at which cells were half-saturated with anti-PD-L1 mAb was estimated by the above binding data representing the estimated dissociation constant (Kd) and then transformed to create a Scatchard plot using GraphPad Prism (version 7; Prism Software Inc., San Diego, USA).

Sample deglycosylation in IHC assay: Formalin-fixed paraffin-embedded (FFPE) tissue sections were incubated at 40° C. overnight and then at 58-65° C. for 1-3 hr, deparaffinized with xylene and ethanol, and hydrated in distilled water. Antigen retrieval was performed with 10 mM citric acid (pH 6.0) in the microwave for 10 min (1000 W for 2 min and 200 W for 8 min) and cooled at room temperature for 60 min. After washing twice with PBS, tissue sections were incubated with 1× glycoprotein denaturing buffer at room temperature for 3 hr, washed with PBS four times, treated without or with PNGase F (5%) containing PBS at 37° C. overnight (12-18 hr), and subjected to IHC staining. In brief, sections were then blocked with 3% H₂O₂/methanol for 10 min at room temperature and washed with PBS three times. Normal scrum (10%) in PBS was added to the sections for 30 min in a humid chamber at room temperature. After wiping off normal serum, PD-L primary antibodies (1:100; Abcam, ab205921, clone 28-8 mAb) were added to the sections in a humid chamber at 40° C. overnight, washed with PBS three times, and incubated with an anti-rabbit secondary antibody (1:200) for 1 hr in a humid chamber at room temperature. Sections were then washed with PBS three times and peroxidase conjugated avidin biotin complex (1:100) was added for 1 hr in a humid chamber at room temperature. After washing with PBS three more times, sections were incubated with AEC chromogen substrate for 5-10 min, washed with distilled water three times, counterstained with Mayer's hematoxylin for 30 sec, washed again with distilled water three times, and mounted with aqua-mount from Lemer Laboratories Inc.

Validation of IHC staining: Validation of IHC assay was performed according to all relevant guidelines from the College of American Pathologists Pathology and Laboratory Quality Center (Fitzgibbons et al., 2014). To set optimal cutoff values, PD-L1 IHC staining was performed using PD-L1 mAb clone 28-8 in the lung cancer tumor microarray (Biomax, #NSC151) to verify the IHC performance. Samples displayed different percentages of the stained cells, ranging from negative (0%) to strongly positive (100%) staining of the tumor cells in the validation set. These percentages in staining obtained from the validation set were applied to other clinical samples described in the manuscript. All staining procedures performed resulted in a characteristic tumor cell pattern of PD-L1 membrane staining.

Evaluation of IHC staining: Two pathologists were tasked with evaluating IHC results independently using an established semi-quantitative approach to assess a Histoscore (H-score) (Detre et al., 1995), which was calculated by both the intensity of staining and the TPS defined by the percentage of PD-L1 positive cells in tumor cells. In brief, for H-score assessment performed as previously described (Wang et al., 2018), 10 fields were randomly chose at 400× magnification, and scored the staining intensity in the malignant cell as 0, 1, 2, or 3 for the presence of negative, weak, intermediate, and strong red staining, respectively. Then the total number of cells were counted in each field and the number of cells stained at each intensity, and calculated the average percentage of positive cells using the following formula: H-score=[1×(% of cells stained at intensity category 1)+2×(% of cells stained at intensity category 2)+3×(% of cells stained at intensity category 3)]. The final H-score ranging from 0 to 300 was obtained for each staining and the average of H-score for all the cases was calculated. Cases with H-score higher than average were regarded as high expression and those with H-score equal or less than average as low expression.

Quantification and statistical analysis: Each sample was assayed in triplicate, unless otherwise noted. All error bars denote standard deviation (SD). Statistical analyses were performed using the GraphPad Prism program (version 7; Prism Software Inc., San Diego, USA). Student's t-test was used to compare two groups of independent samples. Two-tailed Wilcoxon signed-rank test was used to compare two groups of matched samples. Pearson correlation test, two-tailed unless otherwise noted, was used to determine the linear correlation between two variables. Log-rank (Mantel-Cox) and Mantel-Haenszel tests were used to evaluate the statistical significance for the comparison of survival curves and hazard ratios. A p value of <0.05 is considered statistically significant. NS, not significant; no statistical methods were used to predetermine sample size.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Abbondanzo et al., 1990.
Allred et al., 1990.
Brown et al., 1990.
Califano et al., Future Oncol. 14, 2415-2431, 2018.
Detre et al., J Clin Pathol. 48, 876-878, 1995.
Fehrenbacher et al., Lancet. 387, 1837-1846, 2016.
Fitzgibbons et al., Arch Pathol Lab Med. 138, 1432-1443, 2014.
Garon et al, N Engl J Med., 2015.
Huang et al., Onco Targets Ther. 9, 5867-5874, 2016.
International Patent Publication No. WO1995001994
International Patent Publication No. WO1998042752
International Patent Publication No. WO2000037504
International Patent Publication No. WO2001014424
Kim and Leahy, Methods Enzymol. 533:259-63, 2013.
Kita et al., Mol Cell Proteomics. 6(8):1437-45, 2007.
Kowanetz et al., Proc Natl Acad Sci USA. 115, E10119-E10126, 2018.
Kulangara et al., Arch Pathol Lab Med. 143, 330-337, 2019.
Ma et al., J Hematol Oncol., 2016.
Maley et al., Anal Biochem. 180(2):195-204, 1989.
Matthew et al., Ann Transl Med. 5(18):375, 2017.
Pardoll, Nat Rev Cancer. 12(4): 252-64, 2012.
Phillips et al., Appl Immunohistochem Mol Morphol. 23, 541-549, 2015.
Shukuya and Carbone, J Thorac Oncol. 11, 976-988, 2016.
Suzuki et al., Glycoconj J. 12(3):183-93, 1995.
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 5,844,905
U.S. Pat. No. 5,885,796
U.S. Pat. No. 8,008,449
U.S. Pat. No. 8,017,114
U.S. Pat. No. 8,119,129
U.S. Pat. No. 8,329,867
U.S. Pat. No. 8,354,509
U.S. Pat. No. 8,735,553
U.S. Patent Publication No. US20110008369
U.S. Patent Publication No. US20140294898
Wang et al., Cancer Cell. 33, 752-769 e758, 2018.

What is claimed is:

1. An in vitro method for detecting the level of an immune checkpoint protein comprising:
(a) obtaining a fixed sample;
(b) deglycosylating proteins in said sample;
(c) contacting said sample with an anti-immune checkpoint protein antibody; and
(d) measuring the binding of the antibody to said immune checkpoint protein, thereby detecting the level of said immune checkpoint protein.

2. The method of claim 1, wherein the fixed sample is a formalin fixed sample or paraformaldehyde fixed sample.

3. The method of claim 2, wherein the formalin fixed sample is further defined as a formalin-fixed paraffin-embedded (FFPE) sample.

4. The method of claims 1-3, wherein the fixed sample is isolated from saliva, blood, urine, normal tissue, or tumor tissue.

5. The method of claims 1-3, wherein the fixed sample is isolated from tumor tissue.

6. The method of claim 1, wherein the fixed sample does not comprise live cells.

7. The method of claim 1, wherein the immune checkpoint protein is PD-L1, PD-L2, TIM-3, B7-H3, B7-H4, VISTA, CD40, PD-1, CTLA-4, or OX-40L.

8. The method of claim 1, wherein the immune checkpoint protein is PD-L1.

9. The method of claim 8, wherein the anti-immune checkpoint protein antibody is an anti-PD-L1 antibody.

10. The method of claim 1, wherein deglycosylating comprises contacting the fixed sample with a deglycosylation enzyme.

11. The method of claim 10, wherein the deglycosylation enzyme removes N-linked glycosylation.

12. The method of claim 10, wherein the deglycosylation enzyme is peptide-N-glycosidase (PNGase F).

13. The method of claim 12, wherein the sample is incubated with PNGase F for 12-16 hours.

14. The method of claim 12, wherein the sample is incubated with PNGase F at a concentration of 1-10%.

15. The method of claim 12, wherein the sample is incubated with PNGase F at a concentration of 5%.

16. The method of claim 1, wherein measuring comprises performing immunoblotting, immunohistochemistry, ELISA, or immunofluorescence.

17. The method of claim 1, wherein measuring comprises performing immunohistochemistry.

18. The method of claim 1, further comprising administering an anti-cancer therapy to a subject identified to have an increased level of the immune checkpoint protein as compared to a control sample.

19. The method of claim 18, wherein the subject has been previously determined to have no or low expression of the immune checkpoint protein using a non-deglycosylated sample.

20. The method of claim 18, wherein the anti-cancer therapy is chemotherapy, radiotherapy, gene therapy, surgery, hormonal therapy, anti-angiogenic therapy or immunotherapy.

21. The method of claim 18, wherein the anti-cancer therapy is immunotherapy.

22. The method of claim 21, wherein the immunotherapy is an immune checkpoint inhibitor.

23. The method of claim 22, wherein the immune checkpoint inhibitor is a PD-L inhibitor.

24. The method of claim 23, wherein the PD-L1 inhibitor is atezolizumab, avelumab, durvalumab, BMS-936559, or CK-301.

25. The method of claim 24, wherein the immune checkpoint inhibitor is a PD-1 inhibitor.

26. The method of claim 25, wherein the PD-1 inhibitor is pembrolizumab or nivolumab.

27. The method of claim 24, wherein the immune checkpoint inhibitor is a CTLA-4 inhibitor.

28. The method of claim 27, wherein the CTLA-4 inhibitor is ipilimumab or tremelimumab.

29. The method of claim 18, wherein the immune checkpoint inhibitor is administered intravenously, intraperitoneally, intratracheally, intratumorally, intramuscularly, endoscopically, intralesionally, percutaneously, subcutaneously, regionally, or by direct injection or perfusion.

30. The method of claim 1, wherein the subject has cancer.

31. The method of claim 30, wherein the cancer is breast cancer, lung cancer, colon cancer, prostate cancer, pancreatic cancer, melanoma, hepatocellular carcinoma, renal cell carcinoma, urothelial cell carcinoma, or Hodgkin's lymphoma.

32. The method of claim 1, wherein the subject is human.

33. A pharmaceutical composition comprising an immune checkpoint inhibitor for use in a subject determined to have an increased level of said immune checkpoint protein according to the method of any one of claims 1-33.

34. The method of claim 33, wherein the immune checkpoint protein is PD-L1, PD-L2, TIM-3, B7-H3, B7-H4, VISTA, CD40, PD-1, CTLA-4, or OX-40L.

35. The method of claim 33, wherein the immune checkpoint protein is PD-L1.

36. The method of claim 33 or 35, wherein the immune checkpoint inhibitor is PD-L1 inhibitor.

37. The method of claim 36, wherein the PD-L1 inhibitor is atezolizumab, avelumab, durvalumab, BMS-936559, or CK-301.

38. A composition comprising an effective amount of an immune checkpoint inhibitor for the treatment of cancer in a subject, wherein the subject has been determined to have an increased level of said immune checkpoint protein according to any one of claims 1-33.

39. The composition of claim 38, wherein the immune checkpoint protein is PD-L1 PD-L2, TIM-3, B7-H3, B7-H4, VISTA, CD40, PD-1, CTLA-4, or OX-40L.

40. The composition of claim 38, wherein the immune checkpoint protein is PD-L1.

41. The composition of claim 38 or 40, wherein the immune checkpoint inhibitor is PD-L1 inhibitor.

42. The composition of claim 41, wherein the PD-L1 inhibitor is atezolizumab, avelumab, durvalumab, BMS-936559, or CK-301.

43. A method of predicting response to an immune checkpoint inhibitor in a subject having cancer comprising measuring the level of the immune checkpoint protein according to the method of any one of claims 1-33 in a sample obtained from said subject, wherein if the sample has increased expression of the immune checkpoint protein, then the patient is predicted to have a favorable response to the immune checkpoint inhibitor.

44. The method of claim 43, wherein the immune checkpoint protein is PD-L1, PD-L2, TIM-3, B7-H3, B7-H4, VISTA, CD40, PD-1, CTLA-4, or OX-40L.

45. The method of claim 43, wherein the immune checkpoint protein is PD-L1.

46. The method of claim 43 or 45, wherein the immune checkpoint inhibitor is a PD-L1 inhibitor.

47. The method of claim 46, wherein the PD-L1 inhibitor is atezolizumab, avelumab, durvalumab, BMS-936559, or CK-301.

48. The method of claim 43, wherein the immune checkpoint inhibitor is a PD-1 inhibitor.

49. The method of claim 48, wherein the PD-1 inhibitor is pembrolizumab or nivolumab.

50. The method of claim 43, wherein the immune checkpoint inhibitor is a CTLA-4 inhibitor.

51. The method of claim 50, wherein the CTLA-4 inhibitor is ipilimumab or tremelimumab.

52. The method of claim 43, wherein measuring comprises performing immunoblotting, immunohistochemistry, ELISA, or immunofluorescence.

53. The method of claim 43, further comprising administering the immune checkpoint inhibitor to the subject predicted to have a favorable response.

54. The method of claim 53, wherein the immune checkpoint inhibitor is administered intravenously, intraperitoneally, intratracheally, intratumorally, intramuscularly, endoscopically, intralesionally, percutaneously, subcutaneously, regionally, or by direct injection or perfusion.

55. A method of treating cancer in a subject comprising administering an effective amount of an immune checkpoint inhibitor to the subject, wherein the subject has been determined to have an increased level of an immune checkpoint protein according to the method of any of claims 1-33.

56. The method of claim 55, wherein the immune checkpoint protein is PD-L1, PD-L2, TIM-3, B7-H3, B7-H4, VISTA, CD40, PD-1, CTLA-4, or OX-40L.

57. The method of claim 55, wherein the immune checkpoint protein is PD-L1.

58. The method of claim 55 or 57, wherein the immune checkpoint inhibitor is PD-L1 inhibitor.

59. The method of claim 58, wherein the PD-L1 inhibitor is atezolizumab, avelumab, durvalumab, BMS-936559, or CK-301.

60. The method of claim 55, wherein the immune checkpoint inhibitor is a PD-1 inhibitor.

61. The method of claim 60, wherein the PD-1 inhibitor is pembrolizumab or nivolumab.

62. The method of claim 55, wherein the immune checkpoint inhibitor is a CTLA-4 inhibitor.

63. The method of claim 62, wherein the CTLA-4 inhibitor is ipilimumab or tremelimumab.

64. The method of claim 55, wherein the immune checkpoint inhibitor is administered intravenously, intraperitoneally, intratracheally, intratumorally, intramuscularly, endoscopically, intralesionally, percutaneously, subcutaneously, regionally, or by direct injection or perfusion.

65. The method of claims 55-64, wherein the subject is human.

66. The method of claim 55-64, wherein the cancer is breast cancer, lung cancer, colon cancer, prostate cancer, pancreatic cancer, melanoma, hepatocellular carcinoma, renal cell carcinoma, urothelial cell carcinoma, or Hodgkin's lymphoma.

67. The method of claim 55-64, further comprising administering an additional anti-cancer therapy.

68. The method of claim 67, wherein the additional anti-cancer therapy is chemotherapy, radiotherapy, gene therapy, surgery, hormonal therapy, anti-angiogenic therapy or immunotherapy.

69. The method of claim 67, wherein the additional anti-cancer therapy is administered concurrently with the immune checkpoint inhibitor.
